(19) United States
(12) Patent Application Publication
(10) Pub. No.: US 2024/0342167 A1
(43) Pub. Date: Oct. 17, 2024

(54) INHIBITORS OF PORCINE REPRODUCTIVE AND RESPIRATORY SYNDROME VIRUS

(71) Applicants: University of Connecticut, Farmington, CT (US); ATOMWISE INC., San Franscisco, CA (US)

(72) Inventors: Young TANG, Willington, CT (US); Antonio GARMENDIA, Storrs, CT (US); Xiuchun TIAN, Storrs, CT (US); Jiaqi ZHU, Willington, CT (US); Denzil BERNARD, Redwood City, CA (US)

(21) Appl. No.: 18/632,021

(22) Filed: Apr. 10, 2024

Related U.S. Application Data
(60) Provisional application No. 63/458,461, filed on Apr. 11, 2023.

(51) Int. Cl. A61K 31/498; A61P 31/14

(52) U.S. Cl. CPC A61K 31/498; A61P 31/14

(57) ABSTRACT

Described herein are compounds of Formula I, wherein the meanings of the substituents are indicated in the description, for modulating a reproductive and respiratory syndrome virus through multiple mechanisms, and to their use as medicaments for the prevention and/or treatment of diseases related to a reproductive and respiratory syndrome virus. Pharmaceutical compositions comprising said compounds of Formula I are also described.

### CROSS-REFERENCE TO RELATED APPLICATIONS

This application claims priority to U.S. Provisional Patent Application No. 63/458,461 filed on Apr. 11, 2023, which is incorporated by reference herein in its entirety.

### FEDERALLY SPONSORED RESEARCH

This invention was made with government support under 2017-67016-26675, and 2022-67016-37126 awarded by the National Institute of Food and Agriculture. The government has certain rights in the invention.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “209670-0002-US02_sequence_listing_xml_10 Apr. 2024.xml,” was created on Apr. 10, 2024, contains 8 sequences, has a file size of 8.00 KB (8,192 bytes), and is incorporated by reference in its entirety into the specification.

### BACKGROUND

Porcine reproductive and respiratory syndrome (PRRS) is one of the most economically significant swine diseases, leading to an annual loss of $664 million to the United States pork industry, a similar loss in China, and over €1.5 billion in Europe. PRRS results in severe reproductive failure in sows and respiratory diseases in growing pigs and is frequently associated with secondary infections resulting in severe clinical manifestations and mortality. The causative agent, porcine reproductive and respiratory syndrome virus (PRRSV) is an enveloped, positive-sense, single-stranded RNA virus of the family Arteriviridae within the order Nidovirales. PRRS was initially detected in central Europe and North American in the late 1980s, with two genotypes (Type 1 and 2) of PRRSV isolated, which shared 60% of genetic similarity. Unfortunately, due to the high genetic, antigenic, and pathogenic heterogeneities, broadly effective vaccines against PRRSV are still lacking.

What are needed are novel anti-viral compounds and methods to mitigate PRRSV's devastating consequences.

### SUMMARY

One embodiment described herein is a method for treating a reproductive and respiratory syndrome by administering to a subject in need thereof a therapeutically effective amount of a compound, or pharmaceutically acceptable salt thereof, of Formula (I):

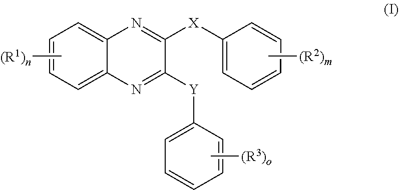

wherein:
X and Y are each independently NR^{a}, O, S, or NR^{a}SO₂;
R¹, at each occurrence, is independently hydrogen, halogen, cyano, nitro, —N(R^{a})₂, —C₁₋₆alkyl, C₁₋₆haloalkyl, —C₂₋₆alkenyl, —C₂₋₆alkynyl, —C₁₋₆alkoxy, or —C₀₋₆alkylCOOR^{a};
R², at each occurrence, is independently hydrogen, halogen, cyano, nitro, —SO₂R^{b}, —N(R^{a})₂, —C₁₋₆alkyl, —C₁₋₆haloalkyl, —C₁₋₆alkoxy, —C₂₋₆alkenyl, —C₂₋₆alkynyl, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R³, at each occurrence, is independently halogen, cyano, nitro, —N(R^{a})₂, —C₁₋₆alkyl, —C₁₋₆haloalkyl, —C₂₋₆alkenyl, —C₂₋₆alkynyl, —C₁₋₆alkoxy, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R^{a}, at each occurrence, is independently hydrogen, —C₁₋₆alkyl, —C₂₋₆alkenyl, —C₂₋₆alkynyl, —C₀₋₆alkyl-C₁₋₆alkoxy, —C₀₋₆alkylCOR^{a}, —C₀₋₆alkyl-COOR^{c}, —C₀₋₆alkyl-C₂₋₆alkenyl, or C₀₋₆alkyl-C₂₋₆alkynyl, R⁶, at each occurrence, is independently —C₁₋₆alkyl, —C₀₋₆alkyl(acyl), —N(R^{C})₂, —NR^{c}-(aryl), —C₀₋₆alkyl-(aryl), —NR^{a}-(heteroaryl), —C₀₋₆alkyl-(aryl), or —C₀₋₆alkyl-(heteroaryl), or —C₁₋₆alkyl-C₁₋₆alkoxy; wherein R₆ is optionally substituted with substituents such as halogen, cyano, nitro, hydroxy, alkoxy, haloalkyl, oxo (C═O), —C₁₋₆alkyl, cycloalkyl such as —C₃₋₇cycloalkyl, a heterocycloalkyl, a heteroaryl, or an aryl;
R^{c}, at each occurrence, is independently hydrogen or —C₁₋₆alkyl,
n, m, and o are each independently an integer of 0, 1, 2, 3, 4, or 5.
   In another aspect, the compound, or a pharmaceutically acceptable salt thereof, has a structure of Formula (Ia):

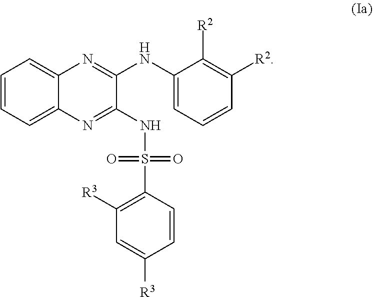

In another aspect, R², at each occurrence, is chloro. In another aspect, R³, at each occurrence, is fluoro or C₁alkyl. In another aspect, the compound, or a pharmaceutically acceptable salt thereof, has a structure of Formula (Ib):

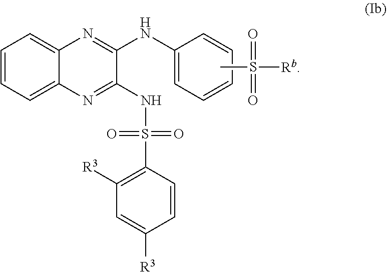

In another aspect, R^{b} is

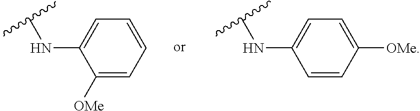 [SMILES: *Nc1ccccc1OC ; "or" ; *Nc1ccc(OC)cc1]

In another aspect, R³, at each occurrence, is fluoro or C₁alkyl. In another aspect, the compound, or pharmaceutically acceptable salt thereof, is selected from the group consisting of:

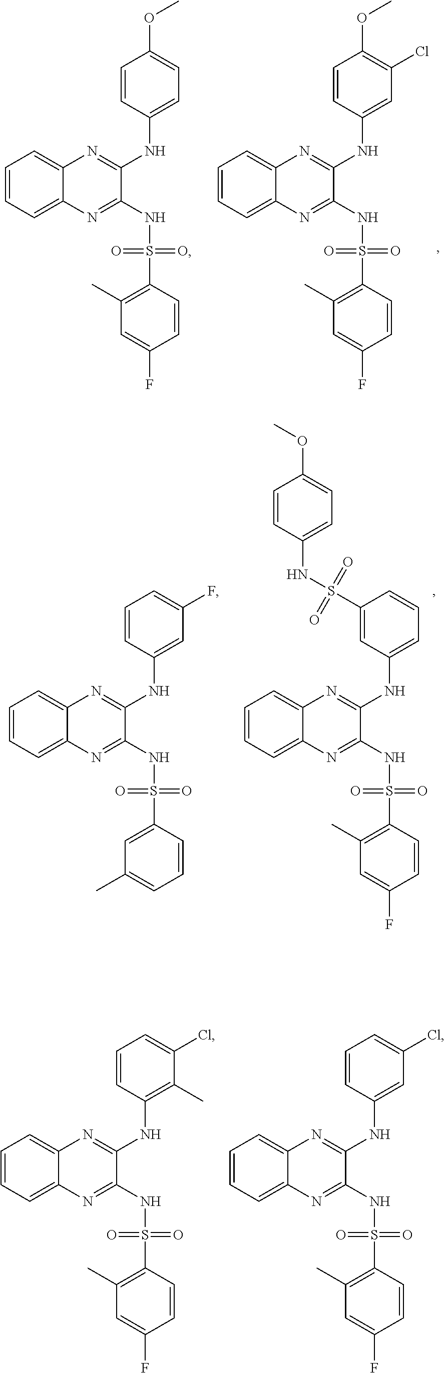

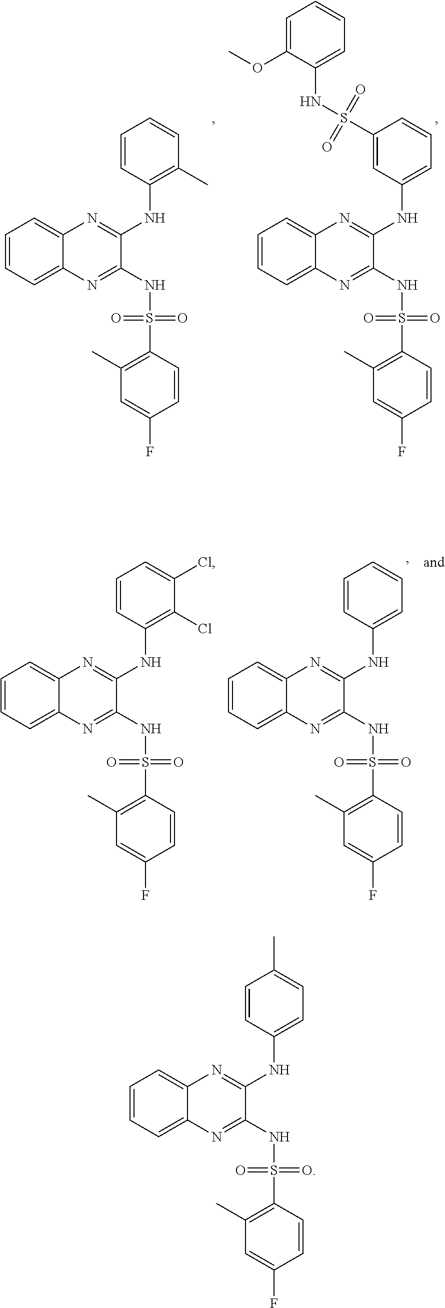

In another aspect, the reproductive and respiratory syndrome is porcine reproductive and respiratory syndrome (PPRS). In another aspect, the reproductive and respiratory syndrome comprises one or more of the following symptoms: fever; lethargy; loss of appetite; vomiting; cough; sneezing; wheezing; labored breathing; high blood pressure; low blood pressure; respiratory distress; depression; cyanosis of the ears, abdomen, or vulva, stillbirths, premature births; abortions; postweaning respiratory diseases; pulmonary edema, or cardiac arrest. In another aspect, decreased function of pulmonary alveolar and intravascular macrophages causes the reproductive and respiratory syndrome. In another aspect, the compound of Formula (I) is administered to the subject prior to, during, and after infection with an arterivirus. In another aspect, the administering is oral, nasal, topical, intravenous, subcutaneous, intramuscular, intravaginal, or intrarectal. In another aspect, administering is performed until the disease or disorder is treated, ameliorated, or at least one symptom is reduced. In another aspect, one or more chemotherapeutic agents, analgesics, anti-infectives, anti-virals, expectorants, decongestants, anti-fever, or other pharmaceutical agents are co-administered. In another aspect, administering the therapeutically effective amount reduces a reproductive and respiratory syndrome virus. In another aspect, the reproductive and respiratory syndrome virus is porcine reproductive and respiratory syndrome virus. In another aspect, the pharmaceutically effective amount is about 0.5 mg/kg to about 2.5 mg/kg of the compound of Formula (I). In another aspect, the subject is a mammal. In another aspect, the subject is a pig.

Another embodiment described herein is a method for treating a reproductive and respiratory syndrome by administering to a subject in need thereof a pharmaceutical composition comprising a therapeutically effective amount of a compound, or pharmaceutically acceptable salt thereof, of Formula (I); and one or more pharmaceutically acceptable carriers or excipients. In another aspect, the one or more pharmaceutically acceptable carriers or excipients comprise buffering agents, solubilizers, solvents, antimicrobial preservatives, antioxidants, suspension agents, a tablet or capsule diluent, or a tablet disintegrant. In another aspect the reproductive and respiratory syndrome is associated with porcine reproductive and respiratory syndrome virus.

Another embodiment described herein is a method for treating porcine reproductive and respiratory (PRR) syndrome by administering to a subject in need thereof a therapeutically effective amount of a compound, or pharmaceutically acceptable salt thereof, of Formula (I):

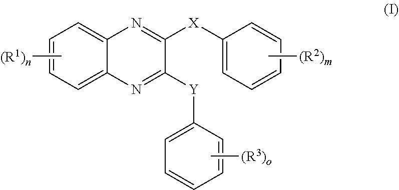

wherein:
X and Y are each independently NR^{a}, O, S, or NR^{a}SO₂;
R¹, at each occurrence, is independently hydrogen, halogen, cyano, nitro, —N(R^{a})₂, —C₁₋₆alkyl, C₁₋₆haloalkyl, —C₂₋₆alkenyl, —C₂₋₆alkynyl, —C₁₋₆alkoxy, or —C₀₋₆alkylCOOR^{a};
R², at each occurrence, is independently hydrogen, halogen, cyano, nitro, —SO₂R^{b}, —N(R^{a})₂, —C₁₋₆alkyl, —C₁₋₆haloalkyl, —C₁₋₆alkoxy, —C₂₋₆alkenyl, —C₂₋₆alkynyl, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R³, at each occurrence, is independently halogen, cyano, nitro, —N(R^{a})₂, —C₁₋₆alkyl, —C₁₋₆haloalkyl, —C₂₋₆alkenyl, —C₂₋₆alkynyl, —C₁₋₆alkoxy, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R^{a}, at each occurrence, is independently hydrogen, —C₁₋₆alkyl, —C₂₋₆alkenyl, —C₂₋₆alkynyl, —C₀₋₆alkyl-C₁₋₆alkoxy, —C₀₋₆alkylCOR^{a}, —C₀₋₆alkyl-COOR^{c}, —C₀₋₆alkyl-C₂₋₆alkenyl, or C₀₋₆alkyl-C₂₋₆alkynyl, R_{b}, at each occurrence, is independently —C₁₋₆alkyl, —C₀₋₆alkyl(acyl), —NR^{c}, —NR^{c}-(aryl), —C₀₋₆alkyl-(aryl), —NR^{a}_(heteroaryl), —C₀₋₆alkyl-(aryl), or —C₀₋₆alkyl-(heteroaryl), or —C₁₋₆alkyl-C₁₋₆alkoxy; wherein R₆ is optionally substituted with substituents such as halogen, cyano, nitro, hydroxy, alkoxy, haloalkyl, oxo (C═O), —C₁₋₆alkyl, cycloalkyl such as —C₃₋₇cycloalkyl, a heterocycloalkyl, a heteroaryl, or an aryl;
R^{c}, at each occurrence, is independently hydrogen or —C₁₋₆alkyl,
n, m, and o are each independently an integer of 0, 1, 2, 3, 4, or 5.
   In another aspect, the compound, or a pharmaceutically acceptable salt thereof, has a structure of Formula (Ia):

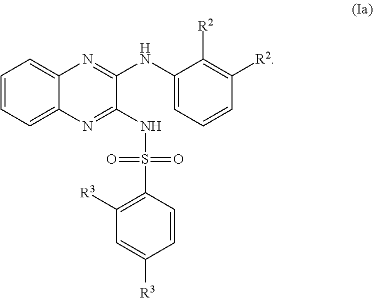

In another aspect, R², at each occurrence, is chloro. In another aspect, R³, at each occurrence, is fluoro or C₁alkyl. In another aspect, the compound, or a pharmaceutically acceptable salt thereof, has a structure of Formula (Ib):

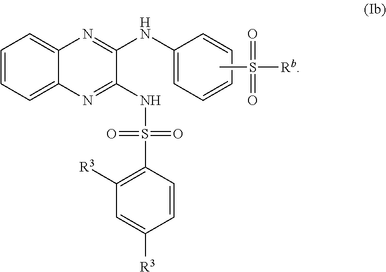

In another aspect, R^{b} is

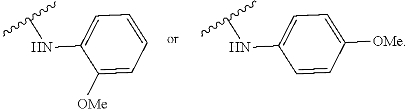 [SMILES: *Nc1ccccc1OC ; *Nc1ccc(OC)cc1]

In another aspect, R³, at each occurrence, is fluoro or C₁alkyl. In another aspect, the compound, or pharmaceutically acceptable salt thereof, is selected from the group consisting of:

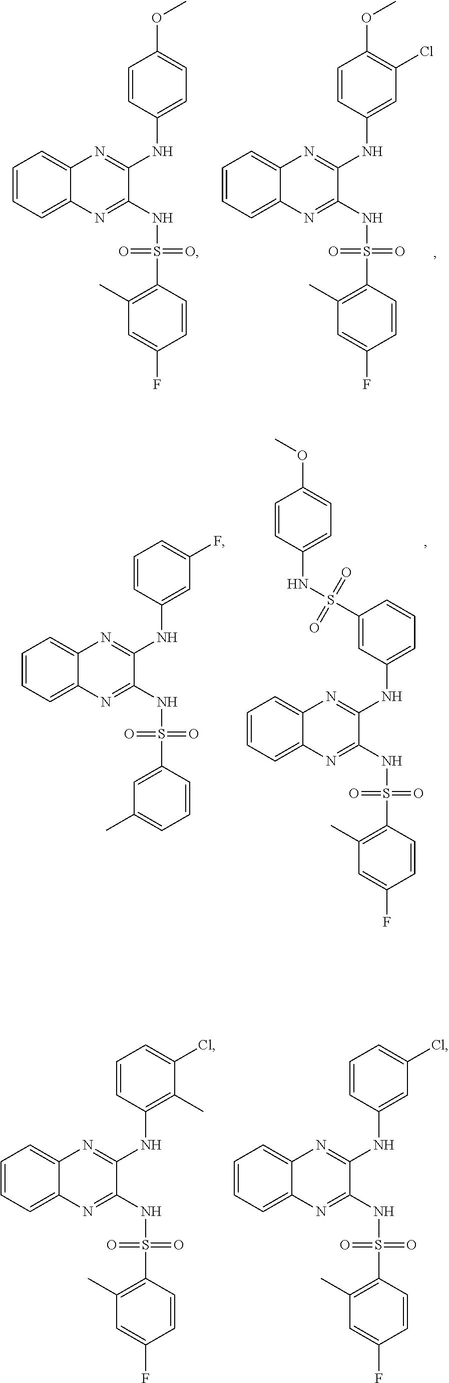 [SMILES: COc1ccc(Nc2nc3ccccc3nc2NS(=O)(=O)c2ccc(F)cc2C)cc1 ; COc1ccc(Nc2nc3ccccc3nc2NS(=O)(=O)c2ccc(F)cc2C)cc1Cl ; Cc1cccc(S(=O)(=O)Nc2nc3ccccc3nc2Nc2cccc(F)c2)c1 ; COc1ccc(NS(=O)(=O)c2cccc(Nc3nc4ccccc4nc3NS(=O)(=O)c3ccc(F)cc3C)c2)cc1 ; Cc1cc(F)ccc1S(=O)(=O)Nc1nc2ccccc2nc1Nc1cccc(Cl)c1C ; Cc1cc(F)ccc1S(=O)(=O)Nc1nc2ccccc2nc1Nc1cccc(Cl)c1]

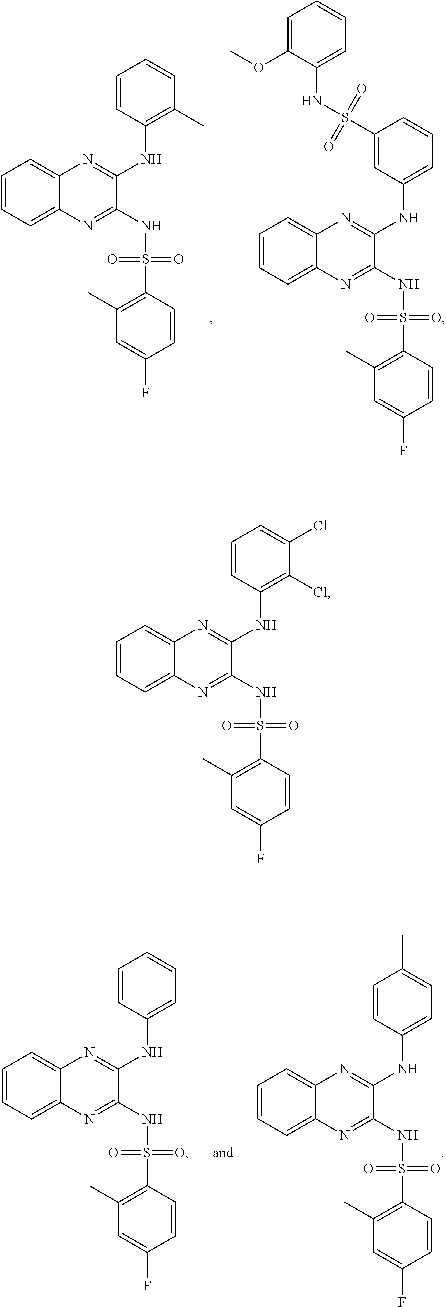 [SMILES: Cc1ccccc1Nc1nc2ccccc2nc1NS(=O)(=O)c1ccc(F)cc1C ; COc1ccccc1NS(=O)(=O)c1cccc(Nc2nc3ccccc3nc2NS(=O)(=O)c2ccc(F)cc2C)c1 ; Cc1cc(F)ccc1S(=O)(=O)Nc1nc2ccccc2nc1Nc1cccc(Cl)c1Cl ; Cc1cc(F)ccc1S(=O)(=O)Nc1nc2ccccc2nc1Nc1ccccc1 ; Cc1ccc(Nc2nc3ccccc3nc2NS(=O)(=O)c2ccc(F)cc2C)cc1]

In another aspect, the porcine reproductive and respiratory syndrome comprises one or more of the following symptoms: fever; lethargy; loss of appetite; vomiting; cough; sneezing; wheezing; labored breathing; high blood pressure; low blood pressure; respiratory distress; depression; cyanosis of the ears, abdomen, or vulva, stillbirths, premature births; abortions; postweaning respiratory diseases; pulmonary edema, or cardiac arrest. In another aspect, decreased function of pulmonary alveolar and intravascular macrophages causes the porcine reproductive and respiratory syndrome. In another aspect, the compound of Formula (I) is administered to the subject prior to, during, and after infection with an arterivirus. In another aspect, the administering is oral, nasal, topical, intravenous, subcutaneous, intramuscular, intravaginal, or intrarectal. In another aspect, administering is performed until the disease or disorder is treated, ameliorated, or at least one symptom is reduced. In another aspect, one or more chemotherapeutic agents, analgesics, anti-infectives, anti-virals, expectorants, decongestants, anti-fever, or other pharmaceutical agents are co-administered. In another aspect, administering the therapeutically effective amount reduces porcine reproductive and respiratory syndrome (PRRS) virus. In another aspect, the pharmaceutically effective amount is about 0.5 mg/kg to about 2.5 mg/kg of the compound of Formula (I). In another aspect, the subject is a mammal. In another aspect, the subject is a pig.

Another embodiment described herein is a method for treating porcine reproductive and respiratory syndrome (PPRS) by administering to a subject in need thereof a pharmaceutical composition comprising a therapeutically effective amount of a compound, or pharmaceutically acceptable salt thereof, of Formula (I); and one or more pharmaceutically acceptable carriers or excipients. In another aspect, the one or more pharmaceutically acceptable carriers or excipients comprise buffering agents, solubilizers, solvents, antimicrobial preservatives, antioxidants, suspension agents, a tablet or capsule diluent, or a tablet disintegrant.

### BRIEF DESCRIPTION OF THE DRAWINGS

FIG. 1A-F show the screen of new compounds that block PRRSV/CD163 interaction. FIG. 1A shows a schematic Diagram for the BiFC assay constructs between CD163 SRCR5 or PRRSV Envelope Proteins GP2a/GP4 and the Fragments of Venus Protein VN155(I152L) or VC155), respectively. FIG. 1B shows BiFC screening images for select positive compounds that inhibit the PPI between SRCR5-VN and GP2a-VC proteins. NC: DMSO Ctrl, Bar=250 μm. FIG. 1C shows relative fluorescence intensity of the SRCR5/GP2a BiFC screening results for all the 20 positive compounds, Mean±SD, n=3. FIG. 1D shows a comparison for the relative fluorescence intensity of the SRCR5/GP2a and SRCR5/GP4 BiFC screening results for the 20 positive compounds, Mean±SD, n=3. FIG. 1E shows BiFC screening images for select positive compounds that inhibit the PPI between SRCR5-VN and GP4-VC proteins. NC: DMSO Ctrl, Bar=250 μm. FIG. 1F shows chemical structure of the 5 identified strong positive compounds by both BiFC assay.

FIG. 2A-B show that the combined GP2a/SRCR5 and GP4/SRCR5 bifc assays accurately predict potent PRRSV-inhibitory compounds. FIG. 2A shows MTT analysis to the 20 positive compounds in PAMs. NC: DMSO Ctrl, Mean±SD, n=3. FIG. 2B shows qRT-PCR for PRRSV in total RNAs extracted from infected PAMs treated with various compounds, values are normalized with GAPDH of PAMs and the average of three strains (NADC30, VR-2332 and Lelystad) are plotted. Bars=mean±SD, n=3. *: P<0.05; **: P<0.01; ***: P<0.001. Mean±SD, n=3. FIG. 2C shows titration assay results for PRRSV in the culture media of PAMs treated as described in (FIG. 2B). Bars=mean±SD, n=3. *: P<0.05; **: P<0.01; ***: P<0.001. Mean±SD, n=3. FIG. 2D shows relative fluorescence intensity of the SRCR5/GP2a BiFC assay for B7, B7-A4, E10, F9 and F12 at different concentrations. Mean±SD, n=3. P values are calculated by one-way ANOVA and letters on the top of bars indicate significant differences in Tukey post-hoc test. FIG. 2E shows MTT analysis to B7, B7-A4, E10, F9 and F12 at different concentrations in PAMs. NC: DMSO Ctrl. Mean±SD, n=3. FIG. 2F shows titration for PRRSV in the culture media from infected PAMs treated with B7, B7-A4, E10, F9 and F12 at different concentrations. Bars=Mean±SD, n=3. Mean±SD, n=3. P values are calculated by one-way ANOVA and letters on the top of bars indicate significant differences in Tukey post-hoc test.

FIG. 3A-D shows validating of physical interactions between the compounds and SRCR5. FIG. 3A shows molecular docking (Top) and PLIP (Table) analyses depicting two potential interaction sites between compounds B7 and CD163-SRCR5 domain. FIG. 3B shows residues in CD163-SRCR5 with potential interactions between B7 (yellow highlighted), residues forming potential hydrogen bonds with B7 are marked with green. The amino acids within the LBP and loop 5-6 regions are underlined. FIG. 3C shows 2-Dimensional ligand-protein interaction diagram by LigPlot+ showing the hydrogen bond and hydrophobic interactions of B7 with the SRCR5. FIG. 3D shows molecular docking analyses by ZDOCK depicting the potential conformation of PPIs between SRCR5 with GP4 (Left) or GP2a (Right). B7 was placed in position 1 (Boxes) to show the potential disturbing of both conformations. FIG. 3E shows molecular docking analyses by ZDOCK depicting the PPI conformation between SRCR5 with both GP4 and GP2a. B7 was placed in position 2 (Box) to show the potential disturbing of this conformation. FIG. 3F shows MST analysis of GFP-fused SRCR5 thermal dynamic association with ligand B7. Values represent Mean, N=3. FIG. 3G shows MST analysis of GFP-fused SRCR5 thermal dynamic association with ligand B7-A4, E10 and F9. Values represent Mean, N=3. FIG. 3H shows MST analysis of GFP-fused SRCR5 thermal dynamic association with ligand E3. Values represent Mean, N=3.

FIG. 4A-C show evaluating compound structure-activity relationship for anti-PRRSV infection. FIG. 4A shows molecular structures of B7 and B7-A4 analogues. FIG. 4B shows qRT-PCR for PRRSV in total RNAs extracted from infected PAMs treated with B7 and B7-A4 analogues. Values are normalized with GAPDH of PAMs. Bars=mean±SD, n=3. P values are calculated by one-way ANOVA and letters on the top of bars indicate significant differences in Tukey post-hoc test. FIG. 4C shows titration assay results for PRRSV in the culture media of PAMs treated as described in (FIG. 4 B). Bars=mean±SD, n=3. Mean±SD, n=3. P values are calculated by one-way ANOVA and letters on the top of bars indicate significant differences in Tukey post-hoc test.

FIG. 5A-C show evaluating compound structure-activity relationship for anti-PRRSV infection. FIG. 5A shows molecular structures of additional compounds with similarly to B7. FIG. 5B shows qRT-PCR for PRRSV in total RNAs extracted from infected PAMs treated with B7 analogues. Values are normalized with GAPDH of PAMs. Bars=mean±SD, n=3. P values are calculated by one-way ANOVA and letters on the top of bars indicate significant differences in Tukey post-hoc test. FIG. 5C shows titration assay results for PRRSV treated by selected compounds as described in (FIG. 5B). Bars=mean±SD, n=3. Mean±SD, n=3. P values are calculated by one-way ANOVA and letters on the top of bars indicate significant differences in Tukey post-hoc test.

FIG. 6 shows Coomassie staining of the SDS-PAGE gel electrophoresis of the purified GFP-SRCR5 and GFP-SRCR2 proteins, BSA (bovine serum albumin) and GFP were used as expression control.

FIG. 7 shows MST analysis of GFP and GFP-fused SRCR2 thermal dynamic association with ligand B7. Values represent Mean, N=3.

FIG. 8A-B show BiFC assay for F12 at pH 6.0. FIG. 8A shows BiFC images for 5 μM F12 that inhibit the PPI between SRCR5-VN and GP2a-VC proteins. NC: DMSO Ctrl. Bar=250 μm.

FIG. 8B shows relative fluorescence intensity of the SRCR5/GP2a BiFC results for 5 μM F12. Mean±SD, n=3.

FIG. 9 shows relative expression of CD163 in PAMs infected with PRRSV VR-2332 and treated with DMSO or B7 for 24 h.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, all percentages (%) refer to mass (or weight, w/w) percent unless noted otherwise.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the phrases “effective amount” or “a therapeutically effective amount” of a compound described herein refers to an amount of the compound described herein that will elicit the biological or medical response of a subject, for example, reduction or inhibition of an enzyme or a protein activity, or ameliorate symptoms, alleviate conditions, slow or delay disease progression, or prevent a disease, etc. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

In one embodiment, the term “a therapeutically effective amount” refers to the amount of the compound described herein that, when administered to a subject, is effective to at least partially alleviate, prevent and/or ameliorate a condition, or a disorder or viral infections, respiratory conditions, reproductive conditions, congenital respiratory impairments, fevers, lethargy, loss of appetite, stillbirths, premature births, abortions, postweaning respiratory diseases, respiratory distress, depression, cyanosis of the ears, abdomen and vulva, or vomiting. In one embodiment, the term “a therapeutically effective amount” refers to the amount of the compound described herein that, when administered to a cell, or a tissue, or a non-cellular biological material, or a medium, is effective to treat or ameliorate viral infections, cell or tissue death, or protein-protein interactions.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. In one embodiment, the subject is a pig.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “formulation” and “composition” can be used interchangeably and refer to a combination of at least two ingredients. In some embodiments, at least one ingredient may be an active agent or otherwise have properties that exert physiologic activity when administered to a subject. As used herein, “therapeutic composition” and “pharmaceutical composition” can be used interchangeably and refer to a combination of at least two ingredients.

Definitions of specific functional groups and chemical terms are described in more detail herein. The chemical elements are identified in accordance with the Periodic Table of the Elements, CAS version, Handbook of Chemistry and Physics, 75^{th} ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} ed, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} ed, Cambridge University Press, Cambridge, 1987.

As used herein, the term “alkyl” refers to a radical of a straight chain or branched saturated hydrocarbon group having from 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tertiary amyl), and hexyl (C₆) (e.g., n-hexyl).

As used herein, the term “alkylene” refers to a divalent radical of an alkyl group, e.g., —CH₂—, —CH₂CH₂—, and —CH₂CH₂CH₂—.

As used herein, the term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and one or more heteroatoms within the parent chain (“heteroC₁₋₈ alkyl”). In some embodiments, a heteroalkyl group is a saturated group having 1 to 7 carbon atoms and one or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

As used herein, the term “heteroalkylene” refers to a divalent radical of a heteroalkyl group.

As used herein, the terms “alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms.

As used herein, the term “oxo” refers to a carbonyl, specifically a ketone with structure C═O.

As used herein, the term “aryl” refers to a stable, aromatic, mono- or bicyclic ring having the specified number of ring carbon atoms. Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms.

As used herein, the term “heteroaryl” refers to a stable, aromatic, mono- or bicyclic ring radical having the specified number of ring atoms and comprising one or more heteroatoms individually selected from nitrogen, oxygen, or sulfur. The heteroaryl radical may be bonded via a carbon atom or heteroatom. Examples of heteroaryl groups include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, quinolinyl, isoquinolinyl, indolyl, indazolyl, oxadiazolyl, benzothiazolyl, quinoxalinyl, and the like. The related term “heteroaryl ring” likewise refers to a stable, aromatic, mono- or bicyclic ring having the specified number of ring atoms and comprising one or more heteroatoms individually selected from nitrogen, oxygen, or sulfur.

As used herein, the term “carbocyclyl” refers to a stable, saturated, or unsaturated, non-aromatic, mono- or bicyclic (fused, bridged, or spiro) ring having the specified number of ring carbon atoms. Examples of carbocyclyl groups include, but are not limited to, the cycloalkyl groups identified above, cyclobutenyl, cyclopentenyl, cyclohexenyl, and the like. In an embodiment, the specified number is C₃-C₁₂ carbons. The related term “carbocyclic ring” likewise refers to a stable, saturated, or unsaturated, non-aromatic, mono- or bicyclic (fused, bridged, or spiro) ring having the specified number of ring carbon atoms.

As used herein, the term “heterocyclyl” or “heterocycloalkyl) refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur. The heterocyclyl radical may be bonded via a carbon atom or heteroatom. In an embodiment, the specified number is C₃-C₁₂ carbons. Examples of heterocyclyl groups include, but are not limited to, azetidinyl, oxetanyl, pyrrolinyl, pyrrolidinyl, tetrahydrofuryl, tetrahydrothienyl, piperidyl, piperazinyl, tetrahydropyranyl, morpholinyl, perhydroazepinyl, tetrahydropyridinyl, tetrahydroazepinyl, octahydropyrrolopyrrolyl, and the like. The related term “heterocyclic ring” likewise refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur.

As used herein the terms “halo” or “halogen” refers to fluorine (fluoro, —F), chlorine (chloro, —Cl), bromine (bromo, —Br), or iodine (iodo, —I).

As used herein the term “substituted,” whether preceded by the term “optionally” or not, means that one or more hydrogens of the designated moiety are replaced with a suitable substituent.

As used herein, the definition of each expression, e.g., alkyl, m, n, etc., when it occurs more than once in any structure, is intended to be independent of its definition elsewhere in the same structure.

### Pharmaceutical Compositions

Disclosed herein are pharmaceutical compositions comprising:
a therapeutically effective amount of a compound, or pharmaceutically acceptable salt thereof, the compound of Formula (I) having a structure:

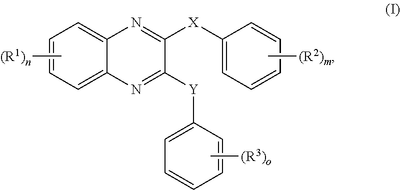

wherein
X and Y are each independently NR^{a}, O, S, or NR^{a}SO₂;
R¹, at each occurrence, is independently hydrogen, halogen, cyano, nitro, —N(R^{a})₂, —C₁₋₆alkyl, C₁₋₆haloalkyl, —C₂₋₆alkenyl, —C₂₋₆alkynyl, —C₁₋₆alkoxy, or —C₀₋₆alkylCOOR^{a};
R², at each occurrence, is independently hydrogen, halogen, cyano, nitro, —SO₂R_{b}, —N(R^{a})₂, —C₁₋₆alkyl, —C₁₋₆haloalkyl, —C₁₋₆alkoxy, —C₂₋₆alkenyl, —C₂₋₆alkynyl, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R³, at each occurrence, is independently halogen, cyano, nitro, —N(R^{a})₂, —C₁₋₆alkyl, —C₁₋₆haloalkyl, —C₂₋₆alkenyl, —C₂₋₆alkynyl, —C₁₋₆alkoxy, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R^{a}, at each occurrence, is independently hydrogen, —C₁₋₆alkyl, —C₂₋₆alkenyl, —C₂₋₆alkynyl, —C₀₋₆alkyl-C₁₋₆alkoxy, —C₀₋₆alkylCOR^{a}, —C₀₋₆alkyl-COOR^{c}, —C₀₋₆alkyl-C₂₋₆alkenyl, or C₀₋₆alkyl-C₂₋₆alkynyl, R^{b}, at each occurrence, is independently —C₁₋₆alkyl, —C₀₋₆alkyl(acyl), —N(R^{c})₂, —NR^{c}-(aryl), —C₀₋₆alkyl-(aryl), —NR^{a}-(heteroaryl), —C₀₋₆alkyl-(aryl), or —C₀₋₆alkyl-(heteroaryl), or —C₁₋₆alkyl-C₁₋₆alkoxy; wherein R_{b} is optionally substituted with substituents such as halogen, cyano, nitro, hydroxy, alkoxy, haloalkyl, oxo (C═O), —C₁₋₆alkyl, cycloalkyl such as —C₃₋₇cycloalkyl, a heterocycloalkyl, a heteroaryl, or an aryl;
R^{c}, at each occurrence, is independently hydrogen or —C₁₋₆alkyl,
n, m, and o are each independently an integer of 0, 1, 2, 3, 4, or 5; and
one or more pharmaceutically acceptable salt thereof, of claim 1; and one or more pharmaceutically acceptable carriers or excipients.

In some embodiments, the pharmaceutically acceptable excipients comprise buffering agents, solubilizers, solvents, antimicrobial preservatives, antioxidants, suspension agents, a tablet or capsule diluent, or a tablet disintegrant. In some embodiments, the compound of Formula I inhibits an interaction between scavenger receptor cysteine-rich domain 5 (SRCR5) of Cluster of Differentiation 163 (CD163) and glycoproteins of an arterivirus. In some embodiments, the compound of Formula I treats, ameliorates the symptoms of, or is prophylactic for an infection by an arterivirus. In some embodiments, the composition comprises about 5 mg to about 400 mg of the compound of Formula I.

Also disclosed herein is a pharmaceutical dosage form comprising a therapeutically effective amount of the pharmaceutical composition described herein.

In some embodiments, the therapeutically effective amount of the pharmaceutical composition comprises about 0.5 mg/kg to about 2.5 mg/kg of the compound of Formula I.

Also disclosed herein are uses of a pharmaceutical composition described herein for treating a reproductive respiratory syndrome by administering to a subject in need thereof an effective amount of the compound of Formula I.

Also disclosed herein are kits comprising one or more dosage forms of the pharmaceutical composition described herein. In some embodiments, the kit further comprises one or more packages, receptacles, delivery devices, labels, and/or instructions for use.

### Pharmaceutical Compositions

The disclosed compositions can be incorporated into pharmaceutical compositions suitable for administration to a subject (such as a patient, which may be a human or non-human). The pharmaceutical composition can be prepared for administration to a subject. Such pharmaceutical compositions can be administered in dosages and by techniques well known to those skilled in the medical, veterinary, and pharmaceutical arts taking into consideration such factors as the age, sex, weight, and condition of the particular subject, and the route of administration.

The pharmaceutical compositions and formulations can include pharmaceutically acceptable carriers. The term “pharmaceutically acceptable carrier,” as used herein, means a non-toxic, inert solid, semi-solid or liquid filler, diluent, encapsulating material, or formulation auxiliary of any type. Exemplary materials that can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their pharmaceutically acceptable salts can be formulated for administration by, for example, injection, inhalation (either through the mouth or the nose), solid dosing, eye drop, in a topical oil-based formulation, implants, oral, buccal, parenteral, or rectal administration. Techniques and formulations generally may be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered, and the form of the composition, will dictate the type of carrier to be used. The composition can be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50% to about 90%.

Suitable lubricants include, but are not limited to, silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition typically is about 5% to about 10%.

Suitable binders include, but are not limited to, polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition typically is about 5% to about 50%.

Suitable disintegrants include, but are not limited to, agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition typically is about 0.1% to about 10%.

Suitable colorants include, but are not limited to, a colorant such as an FD&C dye. When used, the amount of colorant in a systemic or topical composition typically is about 0.005% to about 0.1%.

Suitable flavors include, but are not limited to, menthol, peppermint, and fruit flavors. The amount of flavor(s), when used, in a systemic or topical composition typically is about 0.1% to about 1.0%.

Suitable sweeteners include, but are not limited to, aspartame and saccharin. The amount of sweetener(s) in a systemic or topical composition typically is about 0.001% to about 1%.

Suitable antioxidants include, but are not limited to, butylated hydroxyanisole (“BHA”), butylated hydroxytoluene (“BHT”), and vitamin E. The amount of antioxidant(s) in a systemic or topical composition typically is about 0.1% to about 5%.

Suitable preservatives include, but are not limited to, benzalkonium chloride, methyl paraben and sodium benzoate. The amount of preservative(s) in a systemic or topical composition typically is about 0.01% to about 5%.

Suitable glidants include, but are not limited to, silicon dioxide. The amount of glidant(s) in a systemic or topical composition typically is about 1% to about 5%.

Suitable solvents include, but are not limited to, water, isotonic saline, ethyl oleate, glycerin, hydroxylated castor oils, alcohols such as ethanol, and phosphate buffer solutions. The amount of solvent(s) in a systemic or topical composition typically is from about 0% to about 100%.

Suitable suspending agents include, but are not limited to, AVICEL RC-591 (from FMC Corporation of Philadelphia, PA) and sodium alginate. The amount of suspending agent(s) in a systemic or topical composition typically is about 1% to about 8%.

Suitable surfactants include, but are not limited to, lecithin, Polysorbate 80, and sodium lauryl sulfate, and the TWEEN® detergents. Suitable surfactants include, but are not limited to, those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition typically is about 0.1% to about 5%.

Although the amounts of components in the systemic compositions may vary depending on the type of systemic composition prepared, in general, systemic compositions include about 0.01% to about 50% of an active compound and about 50% to about 99.99% of one or more carriers. Compositions for parenteral administration typically include about 0.1% to about 10% of an active compound and about 90% to about 99.9% of a carrier including a diluent and a solvent.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. In some embodiments, peroral liquid compositions include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions can further include lubricants, colorants, flavors, sweeteners, antioxidants, and/or glidants.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

Pharmaceutical excipients useful for the compositions as described herein include, but are not limited to: acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); alkalizing agents (ammonia solution, ammonium carbonate, diethanolamine, diisopropanolamine, potassium hydroxide, sodium bicarbonate, sodium borate, sodium carbonate, sodium hydroxide, trolamine); antifoaming agents (dimethicone, simethicone); antimicrobial preservatives (benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, ascorbic acid, thimerosal, thymol); antioxidants (ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate); chelating agents (edetate disodium, ethylenediaminetetraacetic acid and salts, edetic acid); coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein); colorants (caramel, red, yellow, black or blends, ferric oxide); complexing agents (ethylenediaminetetraacetic acid and salts (EDTA), edetic acid, gentisic acid ethanolamide, oxyquinoline sulfate); desiccants (calcium chloride, calcium sulfate, silicon dioxide); emulsifying and/or solubilizing agents (acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, emulsifying wax); filtering aids (powdered cellulose, purified siliceous earth); flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); humectants (glycerol, hexylene glycol, sorbitol); plasticizers (e.g., castor oil, diacetylated monoglycerides, diethyl phthalate, glycerol, mono- and di-acetylated monoglycerides, propylene glycol, triacetin, triethyl citrate); polymers (e.g., cellulose acetate, alkyl celluloses, hydroxyalkyl, acrylic polymers and copolymers); solvents (acetone, alcohol, diluted alcohol, amylene hydrate, benzyl benzoate, butyl alcohol, carbon tetrachloride, chloroform, corn oil, cottonseed oil, ethyl acetate, glycerol, hexylene glycol, isopropyl alcohol, methyl alcohol, methylene chloride, methyl isobutyl ketone, mineral oil, peanut oil, propylene carbonate, sesame oil, water for injection, sterile water for injection, sterile water for irrigation, purified water); sorbents (powdered cellulose, charcoal, purified siliceous earth); carbon dioxide sorbents (barium hydroxide lime, soda lime); stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); suspending and/or viscosity-increasing agents (acacia, agar, alginic acid, aluminum monostearate, bentonite, purified bentonite, magma bentonite, carbomer, carboxymethylcellulose calcium, carboxymethylcellulose sodium, carboxymethylcellulose sodium carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, alginate, silicon dioxide, colloidal silicon dioxide, sodium alginate, tragacanth, xanthan gum); sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); surfactants (simethicone); tablet binders (acacia, alginic acid, sodium carboxymethylcellulose, microcrystalline cellulose, dextrin, ethylcellulose, gelatin, liquid glucose, guar gum, hydroxypropyl methylcellulose, methylcellulose, polyethylene oxide, povidone, pregelatinized starch, syrup); tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar); tablet disintegrants (alginic acid, microcrystalline cellulose, croscarmellose sodium, crospovidone, polacrilin potassium, sodium starch glycolate, starch, pregelatinized starch); tablet and/or capsule lubricants (calcium stearate, glyceryl behenate, magnesium stearate, light mineral oil, sodium stearyl fumarate, stearic acid, purified stearic acid, talc, hydrogenated vegetable oil, zinc stearate); thickening agents (gelatin having a bloom strength of 50-100); tonicity agent (dextrose, glycerol, mannitol, potassium chloride, sodium chloride); vehicle: flavored and/or sweetened (aromatic elixir, compound benzaldehyde elixir, iso-alcoholic elixir, peppermint water, sorbitol solution, syrup, tolu balsam syrup); vehicle: oleaginous (almond oil, corn oil, cottonseed oil, ethyl oleate, isopropyl myristate, isopropyl palmitate, mineral oil, light mineral oil, myristyl alcohol, octyl dodecanol, olive oil, peanut oil, persic oil, sesame oil, soybean oil, squalane); vehicle: solid carrier (sugar spheres); vehicle: sterile (bacteriostatic water for injection, bacteriostatic sodium chloride injection); viscosity-increasing (see suspending agent); water repelling agents (cyclomethicone, dimethicone, simethicone); and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol). This list is not meant to be exclusive, but instead merely representative of the classes of excipients and the particular excipients that may be used in oral dosage forms as described herein. See Remington's Essentials of Pharmaceutics, Pharmaceutical Press Publishing Company, London, UK, 1^{st} Edition, 2013, and the Handbook of Pharmaceutical Excipients, 8^{th} Edition, Pharmaceutical Press Publishing Company London, UK, 2017, each of which is incorporated by reference herein for such teachings.

Also described herein are methods for manufacturing a dosage form comprising formulating a composition as described herein comprising sprays, capsules, tablets, elixirs, emulsions, lozenges, suspensions, syrups, pills, lotions, epidermal patches, suppositories, inhalers, or injectables. Any methods known to the art for formulating extracts or active principal ingredients into lotions, soaps, etc. can be utilized.

### Pharmaceutically Acceptable Salts

The disclosed compounds can exist as a pharmaceutically acceptable salt. The term “pharmaceutically acceptable salt” refers to salts or zwitterions of the compounds that are water or oil-soluble or dispersible, suitable for treatment of disorders without undue toxicity, irritation, and allergic response, commensurate with a reasonable benefit/risk ratio and effective for their intended use. The salts can be prepared during the final isolation and purification of the compound or separately by reacting an amino group of the compound with a suitable acid. For example, a compound can be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compound may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compound by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, N,N′-dibenzylethylenediamine, ethylenediamine, ethanolamine, diethanolamine, piperidine, piperazine, and the like.

### Methods of Treatment

In some embodiments, the compounds and pharmaceutical compositions described herein are useful for treating the disorders described herein in a subject in need thereof.

As used herein, the terms “subject” and “patient” may be used interchangeably to refer to any vertebrate including, but not limited to, mammals and humans. In some embodiments, the subject may be a pig. In some embodiments, the subject is undergoing forms of treatment.

The terms “dosage” and “dose” are understood to mean an amount of an active agent that is suitable for administration to a subject in order achieve or otherwise contribute to a therapeutic effect. In some examples, a dosage unit refers to a single dose that is administered to a subject, and is readily handled and packed, remaining as a physically and chemically stable unit dose.

### Administration

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition often is determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of an active compound may be in the range of 1 mg to about 1000 mg of one or more of the compounds described herein. In one aspect, the therapeutically effective amount is about 5 mg to about 400 mg, including all integers and fractions within the range. In another aspect, the therapeutically effective amount is about: 2.5 mg, 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg of one or more of the compounds described herein.

In another embodiment, the therapeutically effective amount of the compounds described herein is about: 0.1 mg/kg, 0.25 mg/kg, 0.5 mg/kg, 0.75 mg/kg, 1 mg/kg, 1.5 mg/kg, 2.0 mg/kg, 2.5 mg/kg, 3 mg/kg, 4 mg/kg, 5 mg/kg, 6 mg/kg, 7 mg/kg, 8 mg/kg, 9, mg/kg, or 10 mg/kg. In one aspect, the therapeutically effect amount of the compounds described herein is about 0.1 mg/kg to about 10 mg/kg. In one aspect, the therapeutically effect amount is about 0.1-1 mg/kg, 0.1-2 mg/kg, 0.1-3 mg/kg, 0.1-4 mg/kg, 0.1-5 mg/kg, 0.1-6 mg/kg, 0.1-7 mg/kg, 0.1-8 mg/kg, 0.1-9 mg/kg, 0.1-10 mg/kg, 0.25-1 mg/kg, 0.25-2 mg/kg, 0.25-3 mg/kg, 0.25-4 mg/kg, 0.25-5 mg/kg, 0.25-6 mg/kg, 0.25-7 mg/kg, 0.25-8 mg/kg, 0.25-9 mg/kg, 0.25-10 mg/kg, 0.5-1 mg/kg, 0.5-2 mg/kg, 0.5-3 mg/kg, 0.5-4 mg/kg, 0.5-5 mg/kg, 0.5-6 mg/kg, 0.5-7 mg/kg, 0.5-8 mg/kg, 0.5-9 mg/kg, 0.5-10 mg/kg, 0.75-1 mg/kg, 0.75-2 mg/kg, 0.75-3 mg/kg, 0.75-4 mg/kg, 0.75-5 mg/kg, 0.75-6 mg/kg, 0.75-7 mg/kg, 0.75-8 mg/kg, 0.75-9 mg/kg, 0.75-10 mg/kg, 1-2 mg/kg, 1-3 mg/kg, 1-4 mg/kg, 1-5 mg/kg, 1-6 mg/kg, 1-7 mg/kg, 1-8 mg/kg, 1-9 mg/kg, or 1-10 mg/kg, including all endpoints, integers and subranges within the disclosed ranges. In one aspect, the therapeutically effective amount is about 0.5 mg/kg to about 2.5 mg/kg including all integers and fractions within the range.

One or more dosage forms of the compositions described herein can be administered, for example, 1×, 2×, 3×, 4×, 5×, 6×, or even more times per day. One or more dosage forms can be administered, for example, for 1, 2, 3, 4, 5, 6, 7 days, or even longer. One or more dosage forms can be administered, for example, for 1, 2, 3, 4 weeks, or even longer. One or more dosage forms can be administered, for example, for 1, 2, 3, 4, 5, 6, 7, 8, 9, 10, 11, 12 months, 1 year, 2, years, 3 years, 4 years, 5 years, over 5 years, a decade, multiple decades, or even longer. One or more dosage forms can be administered at a regular interval until the subject or subject in need thereof, does not require treatment, prophylaxis, or amelioration of any disease or condition including but not limited to a viral infection or the symptoms thereof. In some embodiments, the symptoms may include any one of: fever; lethargy; loss of appetite; vomiting; cough; sneezing; wheezing; labored breathing; high blood pressure; low blood pressure; respiratory distress; depression; cyanosis of the ears, abdomen, or vulva, stillbirths, premature births; abortions; postweaning respiratory diseases; pulmonary edema, or cardiac arrest. In some embodiment, a subject with a reproductive and respiratory syndrome displays at least one symptom associated with a virus, including, but not limited to, fever; lethargy; loss of appetite; vomiting; cough; sneezing; wheezing; labored breathing; high blood pressure; low blood pressure; respiratory distress; depression; cyanosis of the ears, abdomen, or vulva, stillbirths, premature births; abortions; postweaning respiratory diseases; pulmonary edema, or cardiac arrest. In some embodiments, a subject has a virus, and the virus is an arterivirus. In some embodiments, the subject is at risk of contracting, has contracted, or has symptoms associated with porcine reproductive and respiratory syndrome virus (PRRSV). In some embodiments, the symptoms associated with porcine reproductive and respiratory syndrome virus (PRRSV) include, but are not limited to, fever; lethargy; loss of appetite; vomiting; cough; sneezing; wheezing; labored breathing; high blood pressure; low blood pressure; respiratory distress; depression; cyanosis of the ears, abdomen, or vulva, stillbirths, premature births; abortions; postweaning respiratory diseases; pulmonary edema, or cardiac arrest.

In one embodiment, the compositions described herein can be administered as dosage forms in various regimens, including one dose per day (QD), two doses per day (BID), three doses per day (TID), or four times per day (QID) to achieve a total daily dosage. In another embodiment, any of the foregoing doses comprise a total daily dosage.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A method for treating porcine reproductive and respiratory (PRR) syndrome by administering to a subject in need thereof a therapeutically effective amount of a compound, or pharmaceutically acceptable salt thereof, of Formula (I):

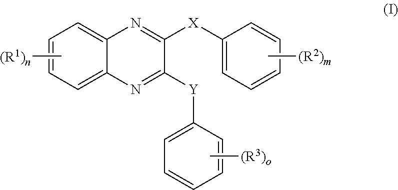

wherein:
X and Y are each independently NR^{a}, O, S, or NR^{a}SO₂;
R¹, at each occurrence, is independently hydrogen, halogen, cyano, nitro, —N(R^{a})₂, —C₁₋₆alkyl, C₁₋₆haloalkyl, —C₂₋₆alkenyl, —C₂₋₆alkynyl, —C₁₋₆alkoxy, or —C₀₋₆alkylCOOR^{a};
R², at each occurrence, is independently hydrogen, halogen, cyano, nitro, —SO₂R_{b}, —N(R^{a})₂, —C₁₋₆alkyl, —C₁₋₆haloalkyl, —C₁₋₆alkoxy, —C₂₋₆alkenyl, —C₂₋₆alkynyl, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R³, at each occurrence, is independently halogen, cyano, nitro, —N(R^{a})₂, —C₁₋₆alkyl, —C₁₋₆haloalkyl, —C₂₋₆alkenyl, —C₂₋₆alkynyl, —C₁₋₆alkoxy, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R^{a}, at each occurrence, is independently hydrogen, —C₁₋₆alkyl, —C₂₋₆alkenyl, —C₂₋₆alkynyl, —C₀₋₆alkyl-C₁₋₆alkoxy, —C₀₋₆alkylCOR^{a}, —C₀₋₆alkyl-COOR^{c}, —C₀₋₆alkyl-C₂₋₆alkenyl, or C₀₋₆alkyl-C₂₋₆alkynyl,
R^{b}, at each occurrence, is independently —C₁₋₆alkyl, —C₀₋₆alkyl(acyl), —N(R^{C})₂, —NR^{c}-(aryl), —C₀₋₆alkyl-(aryl), —NR^{a}-(heteroaryl), —C₀₋₆alkyl-(aryl), or —C₀₋₆alkyl-(heteroaryl), or —C₁₋₆alkyl-C₁₋₆alkoxy; wherein R₆ is optionally substituted with substituents such as halogen, cyano, nitro, hydroxy, alkoxy, haloalkyl, oxo (C═O), —C₁₋₆alkyl, cycloalkyl such as —C₃₋₇cycloalkyl, a heterocycloalkyl, a heteroaryl, or an aryl;
R^{c}, at each occurrence, is independently hydrogen or —C₁₋₆alkyl,
n, m, and o are each independently an integer of 0, 1, 2, 3, 4, or 5.
   Clause 2. The method of clause 1, wherein the compound, or a pharmaceutically acceptable salt thereof, has a structure of Formula (Ia):

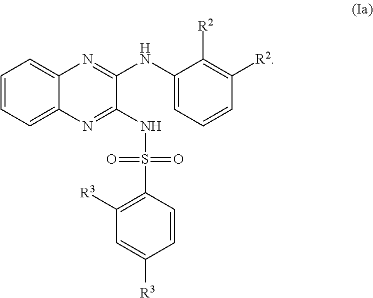

Clause 3. The method of clauses 1 or 2, wherein R², at each occurrence, is chloro.
Clause 4. The method of any one of clauses 1-3, wherein R³, at each occurrence, is fluoro or C₁alkyl.
Clause 5. The method of any one of clauses 1-4, wherein the compound, or a pharmaceutically acceptable salt thereof, has a structure of Formula (Ib):

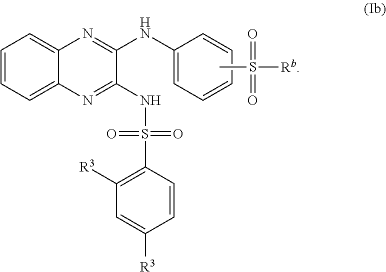

Clause 6. The method of any one of clauses 1-5, wherein R^{b} is

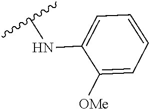 [SMILES: *Nc1ccccc1OC]

or

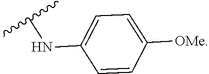 [SMILES: *Nc1ccc(OC)cc1]

Clause 7. The method of any one of clauses 1-6, wherein R³, at each occurrence, is fluoro or C₁alkyl.
Clause 8. The method of any one of clauses 1-7, wherein the compound, or pharmaceutically acceptable salt thereof, is selected from the group consisting of:

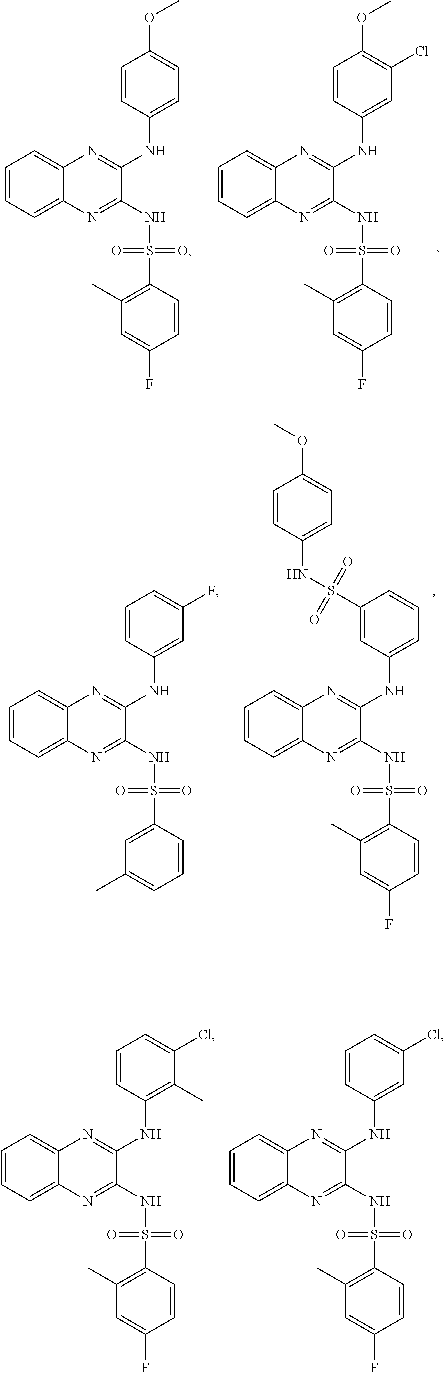

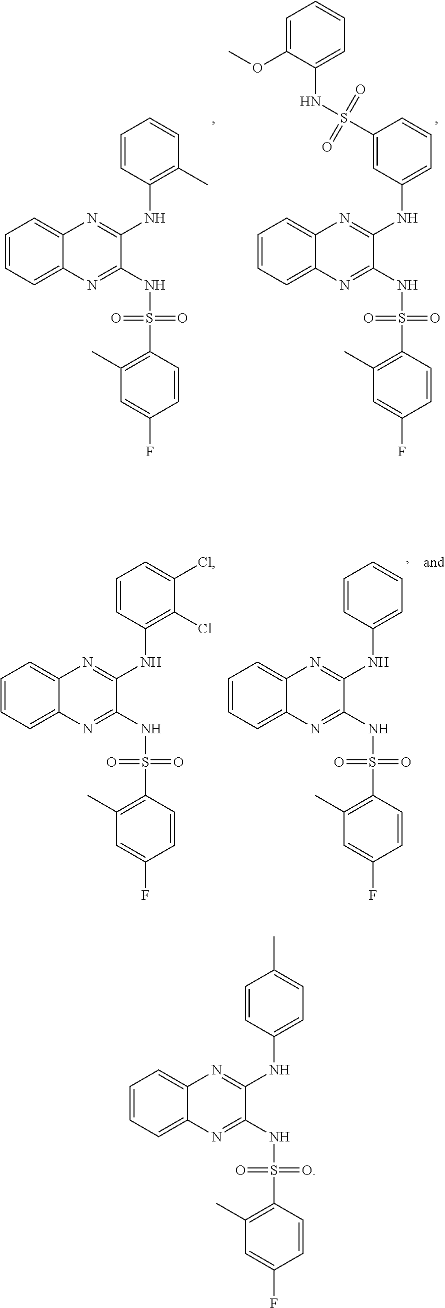

Clause 9. The method of any one of clauses 1-8, wherein the porcine reproductive and respiratory syndrome comprises one or more of the following symptoms: fever; lethargy; loss of appetite; vomiting; cough; sneezing; wheezing; labored breathing; high blood pressure; low blood pressure; respiratory distress; depression; cyanosis of the ears, abdomen, or vulva, stillbirths, premature births; abortions; postweaning respiratory diseases; pulmonary edema, or cardiac arrest.
Clause 10. The method of any one of clauses 1-9, wherein decreased function of pulmonary alveolar and intravascular macrophages causes the porcine reproductive and respiratory syndrome.
Clause 11. The method of any one of clauses 1-10, wherein the compound of Formula (I) is administered to the subject prior to, during, and after infection with an arterivirus.
Clause 12. The method of any one of clauses 1-11, wherein the administering is oral, nasal, topical, intravenous, subcutaneous, intramuscular, intravaginal, or intrarectal.
Clause 13. The method of any one of clauses 1-12, where administering is performed until the disease or disorder is treated, ameliorated, or at least one symptom is reduced.
Clause 14. The method of any one of clauses 1-13, wherein one or more chemotherapeutic agents, analgesics, anti-infectives, anti-virals, expectorants, decongestants, anti-fever, or other pharmaceutical agents are co-administered.
Clause 15. The method of any one of clauses 1-14, wherein administering the therapeutically effective amount reduces porcine reproductive and respiratory syndrome (PRRS) virus.
Clause 16. The method of any one of clauses 1-15, wherein the pharmaceutically effective amount is about 0.5 mg/kg to about 2.5 mg/kg of the compound of Formula (I).
Clause 17. The method of any one of clauses 1-16, wherein the subject is a mammal.
Clause 18. The method of any one of clauses 1-17, wherein the subject is a pig.
Clause 19. A method for treating porcine reproductive and respiratory syndrome (PPRS) by administering to a subject in need thereof a pharmaceutical composition comprising a therapeutically effective amount of a compound, or pharmaceutically acceptable salt thereof, of claim 1; and one or more pharmaceutically acceptable carriers or excipients.
Clause 20. The method of claim 19, wherein, the one or more pharmaceutically acceptable carriers or excipients comprise buffering agents, solubilizers, solvents, antimicrobial preservatives, antioxidants, suspension agents, a tablet or capsule diluent, or a tablet disintegrant.

### Examples

### Chemicals, Cells, Plasmids, and Virus

All chemicals (dissolved in 10 mM in DMSO) were designed and provided by Atomwise, Inc. (CA, USA). Compounds were further diluted in PBS (137 mM NaCl, 2.7 mM KCl, 8 mM Na₂HPO₄, and 2 mM KH₂PO₄, pH 7.4, Gibco, MA, USA) to 200 μM as the working solution. Pulmonary alveolar macrophages (PAMs) were harvested from healthy 4-6-month Landrace/Yorkshire pigs. Briefly, pig lungs were removed from euthanized pigs during necropsy and transferred on ice to the lab. 200 mL warm PBS with 200 U/mL penicillin and 200 μg/mL streptomycin was injected into trachea and reached major bronchi of both sides of the lungs. Lungs were then massaged and bronchoalveolar lavage fluid (BALF) was collected. PAMs were harvested from the BALF by centrifugation at 400×g for 15 min and washed in PBS twice. Fresh PAMs were counted and frozen in 90% FBS and 10% DMSO (Sigma-Aldrich, MO) in liquid nitrogen. PAMs were cultivated in RPMI-1640 (Gibco, MA) supplemented with 10% FBS, 2 mM Glutamax, 0.1 mM MEM Non-Essential Amino Acids, 1 mM sodium pyruvate, 100 U/mL penicillin and 100 μg/mL streptomycin, and 0.5 μg/mL Amphotericin B. SRCR5 domain of porcine CD163 receptor was amplified from cDNA of pig cells and inserted into pBiFC-VN155 (I152L). PRRSV GP2a and GP4 were amplified from reverse-transcribed genome of PRRSV VR2332 strain and cloned into pBiFC-VC155 vector as previously described (Huang et al., Virol J 17, 116, 2020). Porcine CD163 was cloned into pMXs vector (Cell Biolabs, CA, USA). For protein purification, PRRSV GP2a and GP4 were cloned into pET28b-GFP (Addgene, MA, USA).

For CD163-expressing retroviral packaging, pMXs-CD163 was co-transfected with pUMVC and pCMV-VSV-G (Addgene, MA, USA) packaging plasmids into HEK293T cells using Fugene 6 (Promega, WI, USA). Supernatants containing viruses were collected at 48 h and 72 h after transfection. To established CD163 expression Marc145 cell line (CD163-Marc145), Marc145 were cultivated in FP medium (DMEM containing 10% FBS, 2 mM GlutaMAX™ Supplement, 0.1 mM MEM Non-Essential Amino Acids, 50 U/mL and 50 μg/mL Penicillin-Streptomycin) and were incubated with CD163-expressing retrovirus at 32° C. while centrifuging at 650×g for 45 min. The infection was repeated after 24 h. Cells were stained with CD163 Monoclonal Antibody (2A10/11), PE (1:10, Thermo Fisher Scientific, MA, USA) at 24 h after the second infection and fluorescence positive cells were sorted using FACSAria II Cell Sorter (BD, NJ, USA). Genotype I PRRSV strain Lelystad and Genotype II PRRSV strain NADC30 were propagated in PAMs and titrated in CD163-Marc145 cells.

### Artificial Intelligence Molecular Screen (AIMS)

Virtual screening was performed using AtomNet, the first deep neural network for structure-based drug design trained to predict protein-ligand binding affinity. For targeting the interaction between the porcine CD163 and PRRSV glycoprotein (GP2a or GP4), the X-ray structure of CD163-SRCR5 domain (PDBID:5HRJ) was used to define a screening site centered around R561 comprising residues C502, S503, D505, W540, A541, E543, A559, P560, R561, P562, D563, G564, and C566. The Mcule library of commercially available organic small molecule compounds (˜4M v20171018) was prepared and screened, as described previously (Hsieh et al., Miro1 Marks Parkinson's Disease Subset and Miro1 Reducer Rescues Neuron Loss in Parkinson's Models. Cell Metab 30, 1131-1140 e1137, 2019), using an ensemble of protein-ligand conformations. Each of the 4M molecules was scored and ranked by AtomNet, following which a top set of 200 chemically diverse compounds was further inspected for undesirable substructures and molecular properties before 98 compounds were obtained for experimental testing.

### Bimolecular Fluorescence Complementation (BiFC) Assay

HEK293T cells were cultivated in 6-well plates in FP medium and transfected with 1 μg XpBiFC-VN155 (I152L)-SRCR5 and 1 μg pBiFC-VC155-GP2/GP4 using Fugene6. After 4 h, cells were digested with 0.05% Typsin-EDTA (ThermoFisher Scientific, MA, USA)) and passaged to 48-well plates which were coated with EmbryoMax 0.1% Gelatin Solution (Sigma-Aldrich, MO, USA) and preloaded with 5 μM B7 analogues. Fluorescence images were taken at 24 h after transfection and fluorescence intensity was measured by image J.

### Cytotoxicity Assay

PAMs were cultivated in 48-well plates and incubated with 15 μM B7 analogues for 24 h. PAMs treated with 20% DMSO in PBS served as controls. The viability of cells was measured using In Vitro Toxicology Assay Kit (MTT based, Sigma-Aldrich, MO, USA) following the manufacturer's instruction. Briefly, 20 μL Labeling Reagent was added to the cells and incubated at 37° C. for 4 h. And then 200 μL Solubilization Solution was added and incubated at 37° C. overnight. Absorbance at 550 nm was measured using the CLARIOstar Plus plate reader (BMG LABTECH, NC, USA). The cell viability was calculated from the percentage of absorbance of experimental groups to the control groups.

### PRRSV Infection Assay

PAMs were cultivated in 12-well plates and infected with PRRSV NADC30 strain or Lelystad strain at MOI=0.05 for 1 h. Cells were then incubated with 15 μM B7 analogues for 24 h at 37° C. RNAs were extracted from the PAMs for quantitative reverse transcription-PCR (qRT-PCR) using RNeasy Mini Kits (Qiagen, MD, USA) and supernatants of cells were collected PRRSV titration assay.

Quantitative Reverse Transcription-PCR (qRT-PCR)′

RNAs isolated from infected PAMs were reversely transcribed to cDNA using iScript™ cDNA Synthesis Kit (Bio-Rad Laboratories, CA, USA). Specific qRT-PCR primers for ORF7 gene of PRRSV NADC30 and Lelystad strains, and for porcine GAPDH are shown in the Table S2 qRT-PCR reaction was conducted with SYBR Green Supermix (Bimake, TX, USA) using the ABI 7500 Fast Platform (Thermo Fisher Scientific, MA, USA). GAPDH served as the house keeping gene for normalization.

TABLE 1
Primers for qRT-PCR

| Target | Primer | Sequence (5′→3′) | SEQ ID NO |
|---|---|---|---|
| Lelystad- | Forward | AAGATGACATCCGGCACCAC | 1 |
| | Reverse | CCGGCAGCATAAACTCAACCTG | 2 |
| NADC30 | Forward | GGATGGCCAGCCAGTCAATC | 3 |
| | Reverse | TGACGTCATCTTCAGTCGCTAGAG | 4 |
| VR-2332 | Forward | AAACCAGTCCAGAGGCAAGG | 5 |
| | Reverse | GCAAACTAAACTCCACAGTGTAA | 6 |
| GAPDH | Forward | CATCCTGGGCTACACTGAGG | 7 |
| | Reverse | GCTTGACGAAGTGGTCGTTG | 8 |

### PRRSV Titration Assay

CD163-Marc145 were cultivated in 48-well plates and were inoculated with 10-fold serially diluted supernatants from PRRSV infection assay (6 wells for each dilution) at 37° C. for 2 h. The inoculum was replaced by DMEM supplemented with 2% FBS, 2 mM GlutaMAX™ Supplement, 0.1 mM MEM Non-Essential Amino Acids, 50 U/mL and 50 μg/mL Penicillin-Streptomycin. Cells with cytopathic effect were recorded and the Median tissue culture infectious does (TCID50/mL) was calculated using the Reed and Muench method.

### Protein Expression and Purification

The recombinant pET28b-GFP-SRCR2/SRCR5 constructs were transformed into Escherichia coli strain BL21 (DE3) (New England Biolabs, MA, USA). The transformed clones were cultured at 37° C. in LB broth with 50 mg/mL kanamycin, induced by adding 1 mM isopropyl β-D-1-thiogalactopyranoside at optical density of 0.6-0.8, and then incubated at 37° C. for 3 h. For protein purification, cells were pelleted and resuspended in xTractor Buffer containing DNAse I, lysozyme solution, and Protease Inhibitor Cocktail (Takara Bio USA, Inc., CA). The suspension was sonicated and centrifuged. The supernatant was incubated with equilibrated TALON Metal Affinity Resin (Takara Bio USA, Inc., CA, USA) and then were eluted from resin with the elution buffer (pH 7.0, 150 mM imidazole, 50 mM NaH₂PO₄, and 300 mM NaCl). The eluted proteins were concentrated and with buffer exchanged to phosphate-buffered saline (pH 7.4) containing 0.05% Tween-20 (PBST) using Protein Concentrator PES, 10 kD MWCO (Pierce Biotechnology, PA, USA).

### Microscale Thermophoresis (MST) Assay

The purified GFP, GFP-fused SRCR2 or SRCR5 proteins were mixed with serially diluted compounds. The mixtures were loaded to capillaries and the affinity was measured by Monolith NT.115 (NanoTemper Technologies, Inc., CA, USA) at corresponding MST power with the blue channel.

### Statistics Analysis

Data were analyzed by One-way ANOVA with Tukey's post hoc comparison or 2 sample Student's t-test. The figures were presented as mean±sd and p<0.05 was considered statistically significant.

### Results

Screening Compounds that Block PRRSV/CD163 Interaction

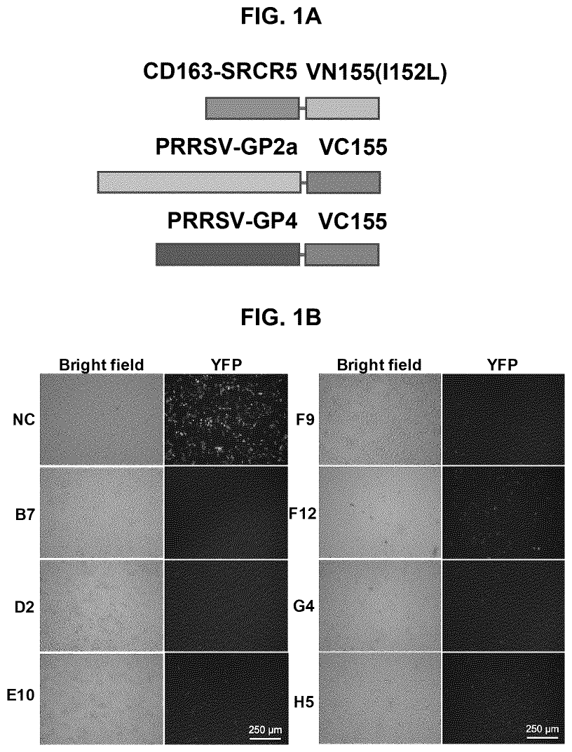

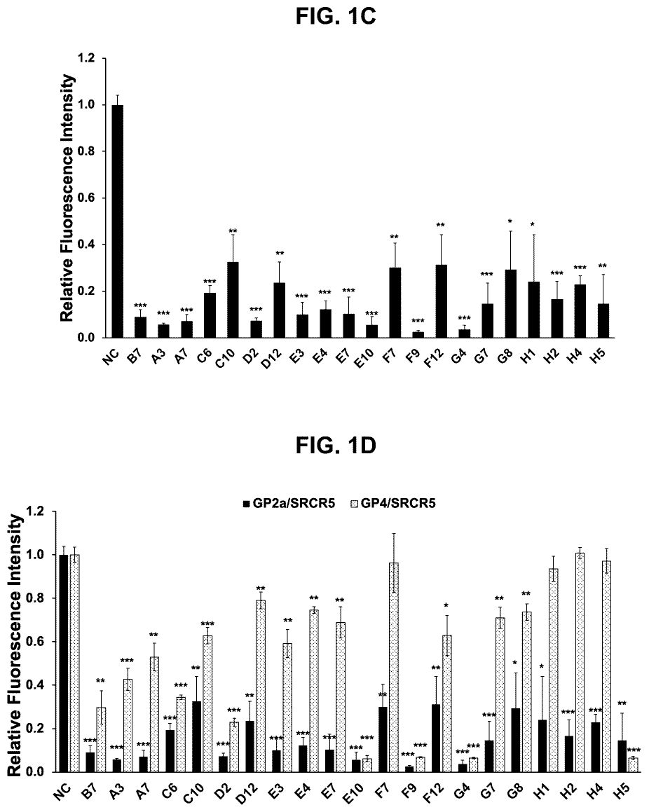

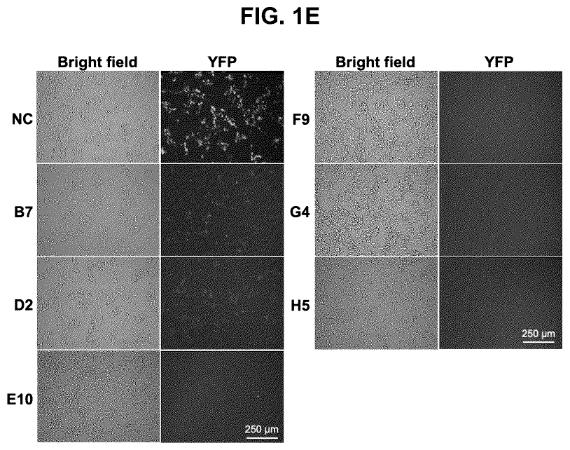

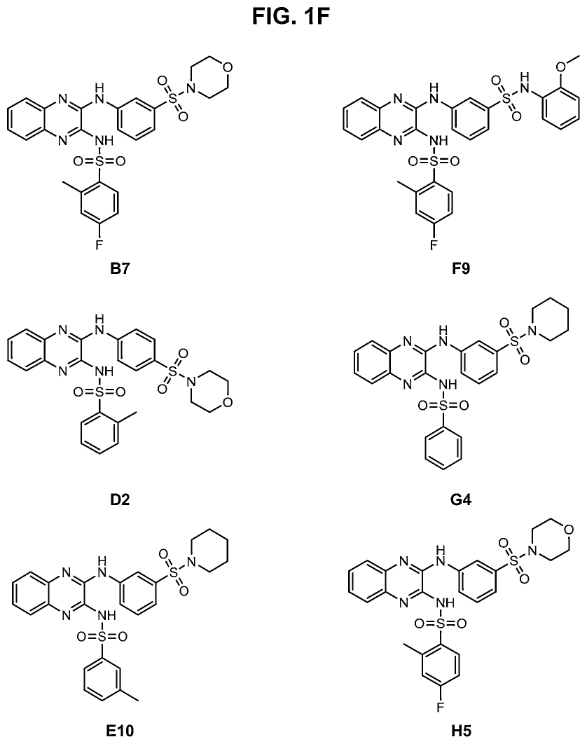

In order to identify SAR with respect to the anti-PRRSV activity of our scaffold, we utilized the artificial intelligence-based AtomNet platform to design a chemically defined library containing 98 compounds (Table 2) with structural similarity to B7 and B7-A4 identified previously. See Huang et al., Virology J. 17(116) (2020). These compounds were predicted to bind to CD163. We first screened the library using a BiFC assay investigating the PPI between PRRSV glycoprotein GP2a and CD163-SRCR5, with B7 and DMSO as the positive- and negative-inhibitory control (PC and NC), respectively (FIG. 1A). Overall, 20 positive hits were identified from the library, which exerted at least 70% reduction of the YFP fluorescence signal compared with the NC (FIG. 1B-C, Table 2). As expected, positive control B7 significantly inhibited the PPI between PRRSV GP2a and CD163 SRCR5 (FIG. 1B-C).

TABLE 2
Compound Activity

| | | |
|---|---|---|
| Average | Std Dev | T test |

| | GP2a | GP4 | GP2a | GP4 | GP2a | GP4 |
|---|---|---|---|---|---|---|
| NC | 1.00 | 1.00 | 0.03 | 0.03 | | |
| PC | 0.09 | 0.30 | 0.08 | 0.08 | 0.0011 | 0.0011 |
| A1 | 0.63 | 0.89 | 0.05 | 0.06 | 0.0364 | 0.0075 |
| A2 | 0.80 | 0.93 | 0.02 | 0.02 | 0.0610 | 0.0610 |
| A3 | 0.06 | 0.43 | 0.05 | 0.05 | 0.0044 | 0.0044 |
| A4 | 0.42 | 0.80 | 0.07 | 0.07 | 0.0245 | 0.0245 |
| A5 | 0.45 | 0.78 | 0.04 | 0.04 | 0.0023 | 0.0023 |
| A6 | 0.55 | 0.67 | 0.03 | 0.03 | 0.0003 | 0.0003 |
| A7 | 0.07 | 0.53 | 0.06 | 0.06 | 0.0057 | 0.0057 |
| A8 | 0.45 | 0.85 | 0.02 | 0.02 | 0.0056 | 0.0056 |
| A9 | 0.16 | 0.55 | 0.06 | 0.05 | 0.0075 | 0.0364 |
| A10 | 0.72 | 0.89 | 0.06 | 0.06 | 0.0853 | 0.0853 |
| A11 | 0.56 | 0.91 | 0.11 | 0.11 | 0.2725 | 0.2725 |
| A12 | 0.66 | 0.96 | 0.03 | 0.03 | 0.1604 | 0.1604 |

| | Average | | Std Dev | | T test | |
|---|---|---|---|---|---|---|
| | GP2a | GP4 | GP2a | GP4 | GP2a | GP4 |
| NC | 1.00 | 1.00 | 0.04 | 0.12 | | |
| PC | 0.15 | 0.19 | 0.02 | 0.03 | 0.0002 | 0.0052 |
| B1 | 0.48 | 0.57 | 0.04 | 0.02 | 0.0001 | 0.0228 |
| B2 | 0.51 | 0.66 | 0.07 | 0.07 | 0.0013 | 0.0217 |
| B3 | 0.53 | 0.71 | 0.09 | 0.05 | 0.0044 | 0.0375 |
| B4 | 0.52 | 0.66 | 0.18 | 0.06 | 0.0357 | 0.0234 |
| B5 | 0.45 | 0.60 | 0.04 | 0.07 | 0.0001 | 0.0138 |
| B6 | 0.62 | 0.82 | 0.16 | 0.07 | 0.0445 | 0.1125 |
| B7_N | 0.36 | 0.32 | 0.01 | 0.01 | 0.0011 | 0.0104 |
| B8 | 0.89 | 0.74 | 0.15 | 0.03 | 0.3334 | 0.0623 |
| B9 | 0.80 | 0.79 | 0.06 | 0.09 | 0.0119 | 0.0770 |
| B10 | 0.90 | 0.79 | 0.10 | 0.08 | 0.2261 | 0.0752 |
| B11 | 0.88 | 0.86 | 0.10 | 0.17 | 0.1651 | 0.3196 |
| B12 | 0.95 | 0.92 | 0.08 | 0.04 | 0.3865 | 0.4012 |

| | | |
|---|---|---|
| Average | Std Dev | T test |

| | GP2a | GP4 | GP2a | GP4 | GP2a | GP4 |
|---|---|---|---|---|---|---|
| NC | 1.00 | 1.00 | 0.20 | 0.08 | | |
| PC | 0.02 | 0.24 | 0.01 | 0.01 | 0.0136 | 0.0034 |
| C1 | 0.41 | 0.81 | 0.03 | 0.07 | 0.0338 | 0.1010 |
| C2 | 0.36 | 1.13 | 0.08 | 0.13 | 0.0195 | 0.2698 |
| C3 | 0.37 | 0.75 | 0.05 | 0.05 | 0.0265 | 0.0144 |
| C4 | 0.43 | 0.87 | 0.02 | 0.05 | 0.0380 | 0.0865 |
| C5 | 0.47 | 0.80 | 0.07 | 0.11 | 0.0337 | 0.0777 |
| C6 | 0.19 | 0.34 | 0.03 | 0.01 | 0.0182 | 0.0084 |
| C7 | 0.65 | 0.85 | 0.06 | 0.07 | 0.0859 | 0.0732 |
| C8 | 0.70 | 0.90 | 0.13 | 0.01 | 0.1100 | 0.1694 |
| C9 | 0.58 | 0.91 | 0.15 | 0.08 | 0.0474 | 0.2199 |
| C10 | 0.33 | 0.63 | 0.12 | 0.04 | 0.0128 | 0.0177 |
| C11 | 0.73 | 0.82 | 0.05 | 0.03 | 0.1356 | 0.0406 |
| C12 | 0.77 | 0.86 | 0.06 | 0.02 | 0.1785 | 0.0832 |

| | | |
|---|---|---|
| Average | Std Dev | T test |

| | GP2a | GP4 | GP2a | GP4 | GP2a | GP4 |
|---|---|---|---|---|---|---|
| NC | 1.00 | 1.00 | 0.18 | 0.08 | | |
| PC | 0.17 | 0.24 | 0.09 | 0.01 | 0.0068 | 0.0034 |
| D1 | 0.92 | 0.85 | 0.17 | 0.06 | 0.6135 | 0.0682 |
| D2 | 0.07 | 0.23 | 0.01 | 0.02 | 0.0125 | 0.0053 |
| D3 | 0.49 | 0.88 | 0.12 | 0.06 | 0.0213 | 0.1001 |
| D4 | 0.67 | 0.82 | 0.05 | 0.07 | 0.0817 | 0.0406 |
| D5 | 0.82 | 0.97 | 0.06 | 0.06 | 0.2269 | 0.6706 |
| D6 | 1.41 | 0.99 | 0.09 | 0.11 | 0.0425 | 0.8934 |
| D7 | 1.32 | 0.80 | 0.08 | 0.10 | 0.0781 | 0.0650 |
| D8 | 1.26 | 1.06 | 0.16 | 0.06 | 0.1371 | 0.2188 |
| D9 | 1.01 | 0.95 | 0.13 | 0.18 | 0.9676 | 0.6535 |
| D10 | 1.27 | 1.02 | 0.05 | 0.04 | 0.1147 | 0.5478 |
| D11 | 1.15 | 1.05 | 0.38 | 0.00 | 0.5831 | 0.0841 |
| D12 | 0.24 | 0.79 | 0.09 | 0.04 | 0.0082 | 0.0636 |

| | | |
|---|---|---|
| Average | Std Dev | T test |

| | GP2a | GP4 | GP2a | GP4 | GP2a | GP4 |
|---|---|---|---|---|---|---|
| NC | 1.00 | 1.00 | 0.22 | 0.03 | | |
| PC | 0.02 | 0.05 | 0.02 | 0.01 | 0.0160 | 0.0000 |
| E1 | 0.70 | 0.83 | 0.05 | 0.06 | 0.1418 | 0.0238 |
| E2 | 0.71 | 0.84 | 0.05 | 0.05 | 0.1446 | 0.0174 |
| E3 | 0.10 | 0.59 | 0.05 | 0.06 | 0.0156 | 0.0087 |
| E4 | 0.12 | 0.74 | 0.04 | 0.02 | 0.0185 | 0.0519 |
| E5 | 0.84 | 0.64 | 0.11 | 0.09 | 0.3437 | 0.0143 |
| E6 | 0.61 | 0.63 | 0.04 | 0.04 | 0.0878 | 0.0002 |
| E7 | 0.10 | 0.69 | 0.07 | 0.07 | 0.0130 | 0.0186 |
| E8 | 0.60 | 0.82 | 0.16 | 0.12 | 0.0714 | 0.1061 |
| E9 | 0.52 | 0.82 | 0.07 | 0.11 | 0.0518 | 0.0869 |
| E10 | 0.06 | 0.06 | 0.04 | 0.01 | 0.0160 | 0.0039 |
| E11 | 0.52 | 0.92 | 0.17 | 0.03 | 0.0457 | 0.0248 |
| E12 | 0.61 | 0.91 | 0.07 | 0.06 | 0.0802 | 0.1136 |

| | | |
|---|---|---|
| Average | Std Dev | T test |

| | GP2a | GP4 | GP2a | GP4 | GP2a | GP4 |
|---|---|---|---|---|---|---|
| NC | 1.00 | 1.00 | 0.09 | 0.02 | | |
| PC | 0.28 | 0.86 | 0.04 | 0.02 | 0.0012 | 0.0008 |
| F1 | 0.90 | 0.98 | 0.20 | 0.05 | 0.4978 | 0.6080 |
| F2 | 0.83 | 0.88 | 0.07 | 0.03 | 0.0554 | 0.0059 |
| F3 | 0.89 | 0.94 | 0.02 | 0.06 | 0.1484 | 0.2195 |
| F4 | 0.84 | 0.86 | 0.13 | 0.02 | 0.1534 | 0.0008 |
| F5 | 1.04 | 0.88 | 0.13 | 0.06 | 0.6700 | 0.0648 |
| F6 | 0.70 | 0.84 | 0.03 | 0.11 | 0.0169 | 0.1209 |
| F7 | 0.30 | 0.96 | 0.10 | 0.14 | 0.0010 | 0.7306 |
| F8 | 0.52 | 0.90 | 0.12 | 0.04 | 0.0074 | 0.0364 |
| F9 | 0.03 | 0.07 | 0.01 | 0.00 | 0.0026 | 0.0044 |
| F10 | 0.61 | 0.89 | 0.09 | 0.10 | 0.0059 | 0.1900 |
| F11 | 0.74 | 0.88 | 0.19 | 0.06 | 0.1335 | 0.0648 |
| F12 | 0.31 | 0.63 | 0.13 | 0.09 | 0.0026 | 0.0110 |

| | | |
|---|---|---|
| Average | Std Dev | T test |

| | GP2a | GP4 | GP2a | GP4 | GP2a | GP4 |
|---|---|---|---|---|---|---|
| NC | 1.00 | 1.00 | 0.01 | 0.02 | | |
| PC | 0.01 | 0.89 | 0.00 | 0.02 | 0.0000 | 0.0008 |
| G1 | 0.61 | 0.84 | 0.12 | 0.11 | 0.0282 | 0.1209 |
| G2 | 0.64 | 0.90 | 0.20 | 0.04 | 0.0856 | 0.0364 |
| G3 | 0.64 | 0.89 | 0.20 | 0.10 | 0.0861 | 0.1900 |
| G4 | 0.04 | 0.06 | 0.02 | 0.00 | 0.0000 | 0.0043 |
| G5 | 0.80 | 0.89 | 0.23 | 0.02 | 0.2680 | 0.0026 |
| G6 | 0.53 | 0.82 | 0.05 | 0.03 | 0.0017 | 0.0029 |
| G7 | 0.15 | 0.71 | 0.09 | 0.05 | 0.0030 | 0.0274 |
| G8 | 0.29 | 0.74 | 0.16 | 0.04 | 0.0167 | 0.0388 |
| G9 | 0.33 | 0.86 | 0.13 | 0.02 | 0.0112 | 0.0022 |
| G10 | 0.13 | 0.80 | 0.13 | 0.11 | 0.0066 | 0.0893 |
| G11 | 0.37 | 0.87 | 0.13 | 0.14 | 0.0120 | 0.2406 |
| G12 | 0.40 | 0.82 | 0.18 | 0.07 | 0.0271 | 0.0269 |

| | | |
|---|---|---|
| Average | Std Dev | T test |

| | GP2a | GP4 | GP2a | GP4 | GP2a | GP4 |
|---|---|---|---|---|---|---|
| NC | 1.00 | 0.88 | 0.32 | 0.03 | | |
| PC | 0.03 | 0.94 | 0.03 | 0.06 | 0.0336 | 0.1980 |
| H1 | 0.24 | 0.94 | 0.20 | 0.06 | 0.0343 | 0.4252 |
| H2 | 0.17 | 1.01 | 0.08 | 0.03 | 0.0404 | 0.9078 |
| H3 | 0.33 | 0.86 | 0.24 | 0.02 | 0.0493 | 0.2970 |
| H4 | 0.23 | 0.97 | 0.04 | 0.06 | 0.0520 | 0.7115 |
| H5 | 0.15 | 0.06 | 0.12 | 0.01 | 0.0316 | 0.0042 |
| H6 | 0.52 | 0.88 | 0.04 | 0.06 | 0.1201 | 0.9531 |
| H7 | 0.43 | 0.84 | 0.03 | 0.11 | 0.0905 | 0.6166 |
| H8 | 0.51 | 0.90 | 0.16 | 0.04 | 0.1037 | 0.5239 |
| H9 | 0.65 | 0.89 | 0.23 | 0.10 | 0.2136 | 0.8427 |
| H10 | 0.62 | 0.88 | 0.21 | 0.06 | 0.1703 | 0.9531 |
| H11 | 0.74 | 0.84 | 0.09 | 0.11 | 0.3027 | 0.6166 |
| H12 | 0.93 | 0.90 | 0.26 | 0.04 | 0.7809 | 0.5239 |

Successful PRRSV infection involves the PPI between CD163 with both glycoproteins GP2a and GP4. To determine if the candidate compounds identified above could inhibit both interactions, we conducted another BiFC assay investigating the PPI between PRRSV GP4 and CD163-SRCR5 domain (FIG. 1A). Out of the 20 positive hits identified above, only 5 (D2, E10, F9, G4, and H5) exhibited more than 70% reduction of the YFP fluorescence signal compared with NC (FIG. 1D-F). Interestingly, these 5 compounds all exerted more than 85% and 90% inhibition in GP2a/SRCR5 and GP4/SRCR5 BiFC assays, respectively (FIG. 1D). Among them, compounds D2 and H5 (FIG. 1F) were found to be structurally identical to those of B7-A2 and B7, respectively as reported previously. See Huang et al., Virology J. 17(116) (2020). The remaining three new positive hits—E10, F9, and G4 all exerted more than 90% inhibition in both assays (FIG. 1D-E). To investigate if the compounds with less than 70% inhibition in GP2a/SRCR5 BiFC assay may exhibit significant potency in GP4/SRCR5 BIFC analysis, we screened the remaining 78 chemicals in the library using GP4/SRCR5 BiFC assay. No additional compound was identified with more than 70% reduction of the fluorescence signal compared to NC (Table 2). Therefore, both GP2a/SRCR5 and GP4/SRCR5 BiFC screening of the library demonstrated a similar trend for compound activities. However, assay screening using the PPI between GP4 and SRCR5 is more productive for identifying compounds with high inhibitory activity.

### The Combined GP2a/SRCR5 and GP4/SRCR5 BiFC Assays Accurately Predict Potent PRRSV-Inhibitory Compounds

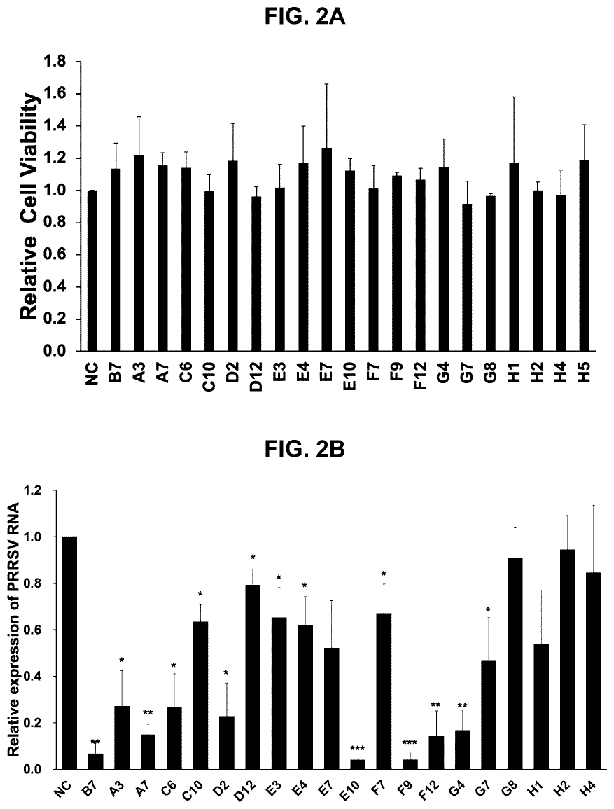

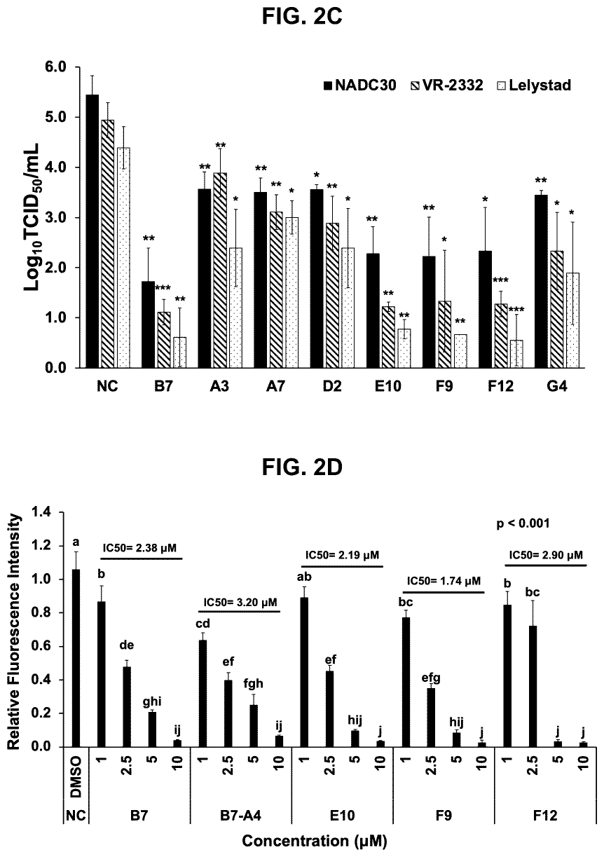

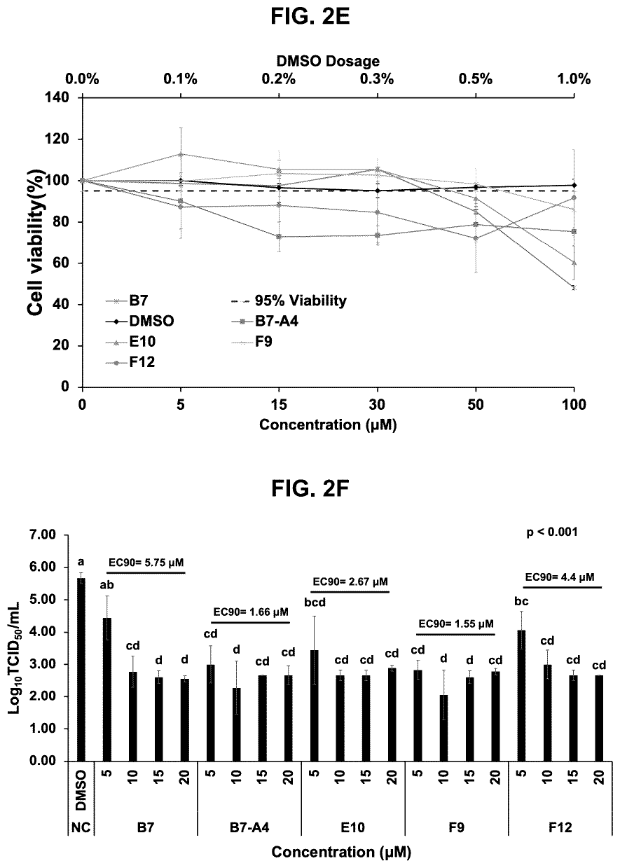

MTT analysis of the inhibitory compounds identified above revealed no obvious cytotoxicity in 48 h for PAMs incubated with the 20 small chemicals at a 15 μM level (FIG. 2A). We then inoculated fresh PAMs with different PRRSV strains including NADC30, VR-2332 (Type 1) and Lelystad (Type 2) followed by the addition of each compound at 15 μM in culture medium immediately after infection (H5 was excluded as it is structurally identical to B7). At 24 h post infection, viral RNAs were extracted from PAMs for the evaluation of viral loads and cell supernatants were collected to measure the viral titer. qRT-PCR revealed that 8 out of the 19 compounds tested showed more than 70% inhibition of viral RNA loads for the average of three viral strains (FIG. 2B). Among these, five compounds A7, E10, F9, F12, and G4 appeared to have the strongest inhibitory effect against infection with more than 85% deduction, and seconded by three other compounds A3, C6, and D2 with 70%-80% decrease (FIG. 2B). This included all the highly positive compounds identified by the GP4/SRCR5 BiFC analysis—D2, E10, F9, and G4 (FIG. 2B). The remaining four compounds (A3, A7, C6, and F12) that exhibited over 70% inhibition of GP2a/SRCR5 BiFC but not GP4/SRCR5 BiFC screening (FIG. 1D) also showed mild (A3 and C6) or strong (A7 and F12) inhibitory effect against PRRSV infection, respectively (FIG. 2B).

The antiviral effects of selected positive compounds were further confirmed by viral titration assay of culture medium harvested from infected PAMs. Similar to the qRT-PCR analysis, treatment by compounds E10, F9, and F12 exhibited strong reduction in the viral titer across different PRRSV strains (3.1-3.3 logs for NADC30, 3.6-3.8 logs for VR-2332 and 3.6-3.9 logs for Lelystad), while compounds A3, A7, D2, and G4 also reduced the viral titer of three strains (1.8-2 logs for NADC30, 1-2.6 logs for VR-2332 and 1.4-2.5 logs for Lelystad) (FIG. 2C). Therefore, compounds that effectively interfered with the GP4/SRCR5 PPI all exerted strong inhibitory potency against PRRSV infection. Of note, although not all compounds effectively interfering with the GP2a/SRCR5 PPI exhibited high potency against viral infection, this BiFC screening identified additional compounds not detected by the GP4/SRCR5 BiFC assay, including F12. Taken together, a combined interrogation to the PPIs of GP4/SRCR5 and GP2a/SRCR5 can facilitate the identification of highly effective compounds against both Type 1/2 PRRSV strains.

### Validation of the Positive Compounds—SRCR5 Molecular Interaction

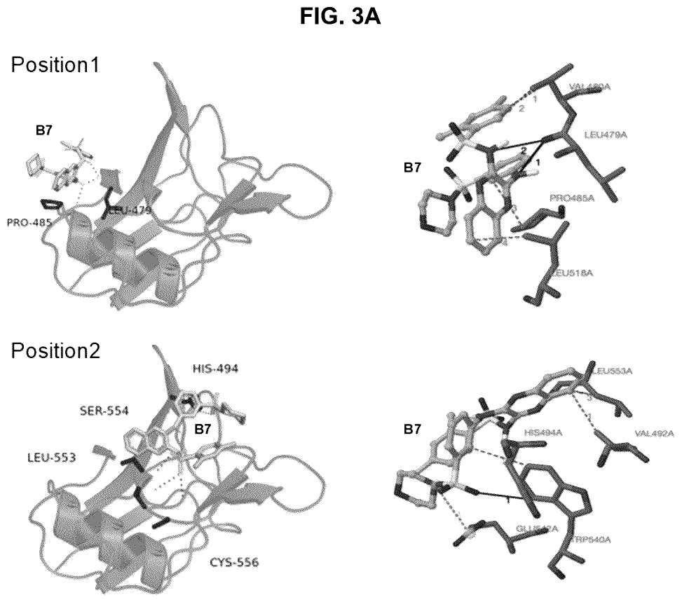

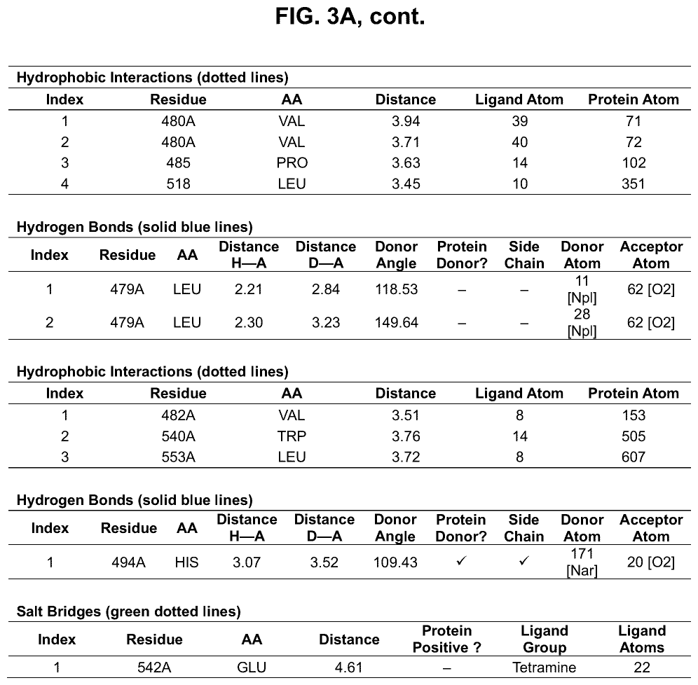

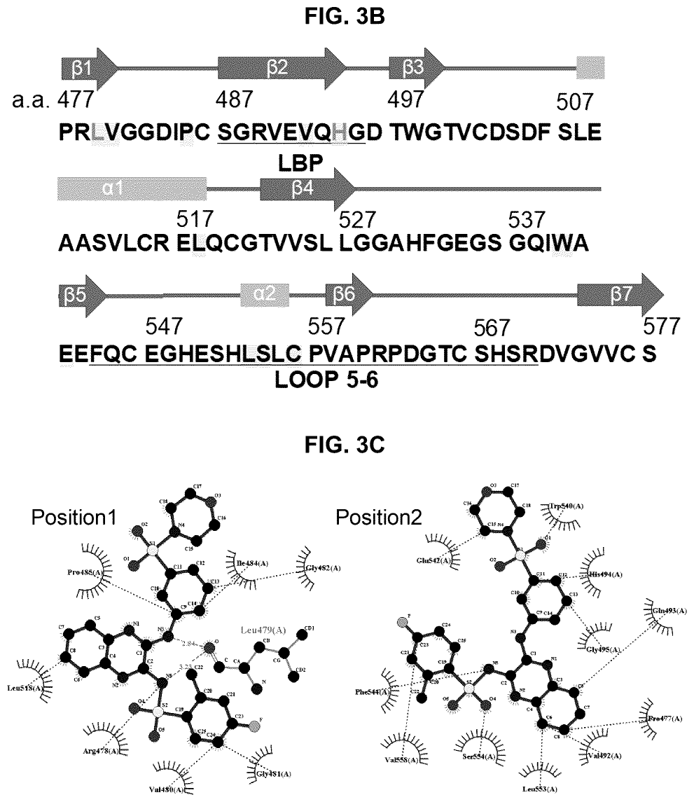

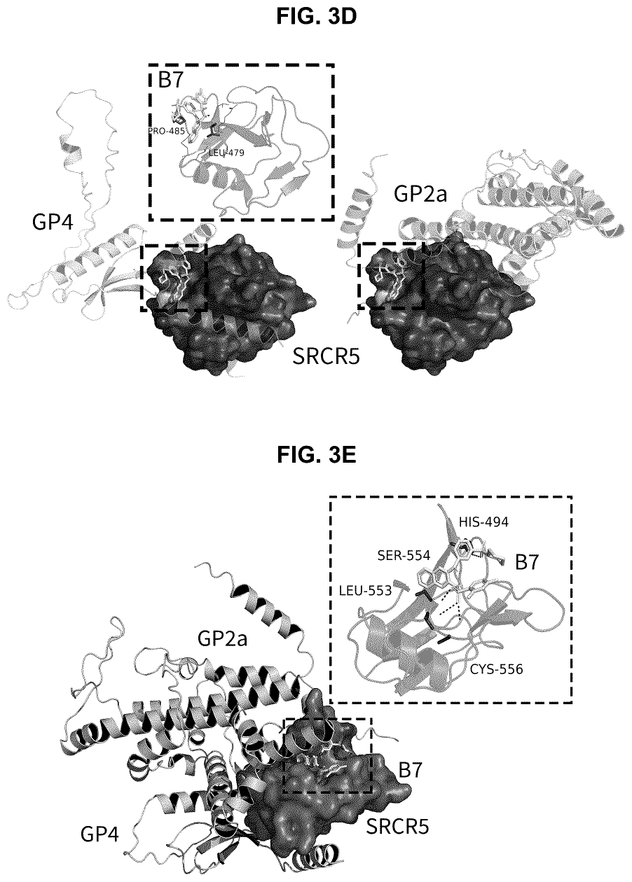

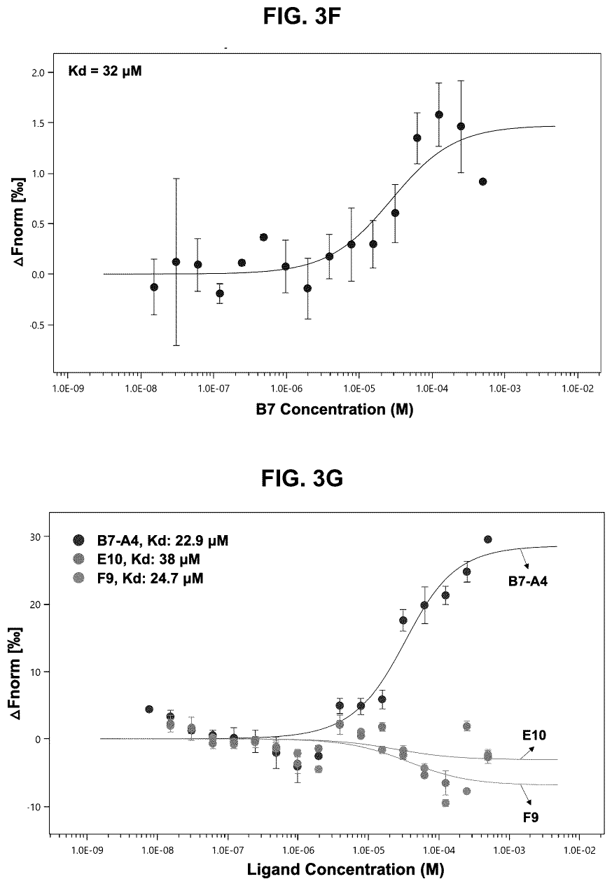

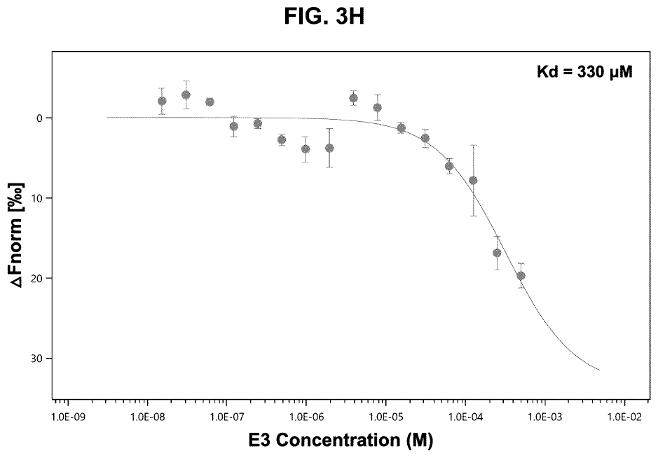

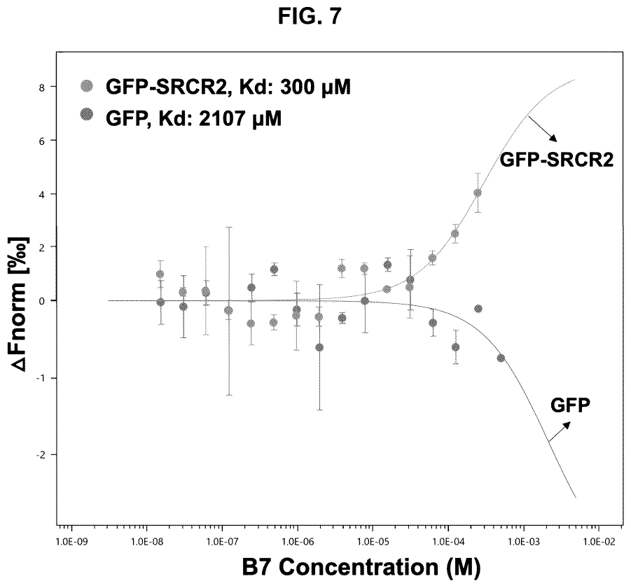

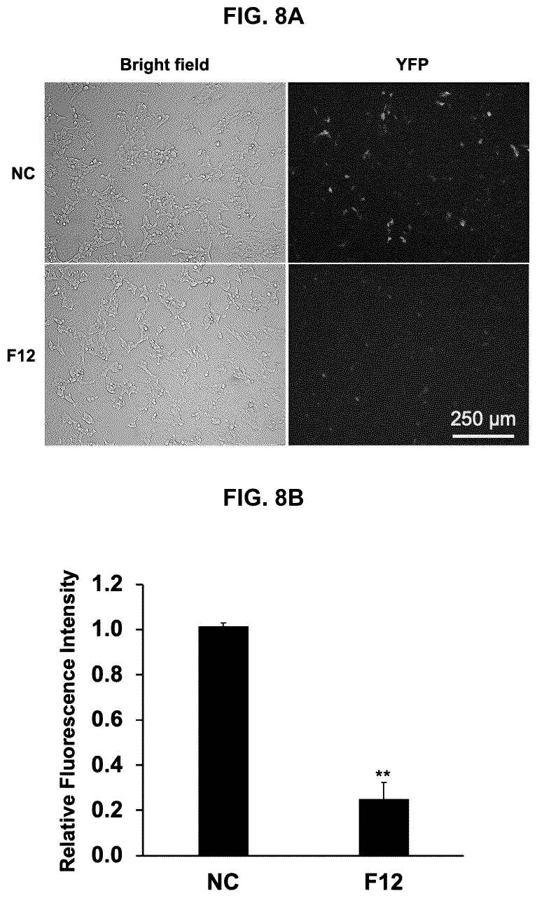

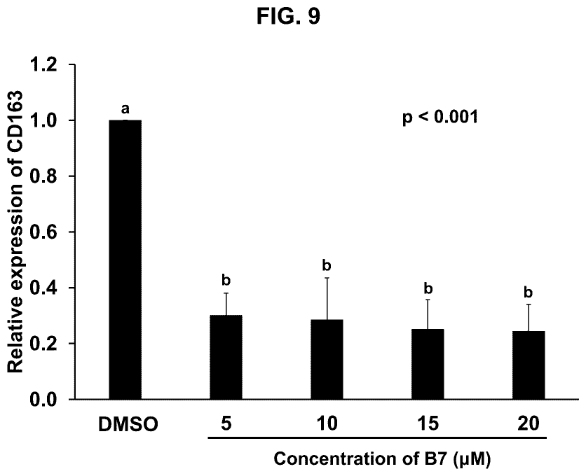

In order to confirm the ligand-receptor physical binding between the small chemicals and CD163, we first performed molecular docking analysis using the prototypical inhibitory compound B7 and the resolved CD163-SRCR5 protein crystal structure. The docking analysis indicated that B7 bind to SRCR5 pocket region and interacts with residues P477 and S554 (FIG. 3A), two residues unique for SRCR5 across the nine CD163-SRCR domains based on the sequence alignment. To determine whether there was physical association between the chemicals and CD163, we expressed GFP-fusion CD163-SRCR5 recombinant protein, with GFP and GFP-fusion CD163-SRCR2 as a control because they do not interact with GP2a and GP4 or bind to B7 (FIG. 7). The microscale thermophoresis (MST) assay was used to evaluate the direct association between B7 and the GFP-fusion proteins. The MST analysis demonstrated a physical binding between B7 and the GFP-SRCR5 with a dissociation constant (K_{d}) value of 32 μM (FIG. 3B). However, little or no association was detected between B7 and GFP-SRCR2 or GFP (FIG. 8). Similar ligand-protein interactions were found between GFP-SRCR5 and compounds B7-A4, E10, and F9, with similar KD values as B7 (FIG. 3C). These K_{d} values correlated well with their demonstrated potency against PRRSV infection (FIG. 2B-C). On the contrary, compound E3 which showed low activity against PRRSV infection (FIG. 2B) exhibited a K_{d} value 10 times greater than B7 (FIG. 3D). Therefore, these data indicate that the positive compounds including B7 and its analogues physically target CD163-SRCR5 domain to exert their function against PRRSV infection.

### SAR Evaluation of the Compounds for Anti-PRRSV Function

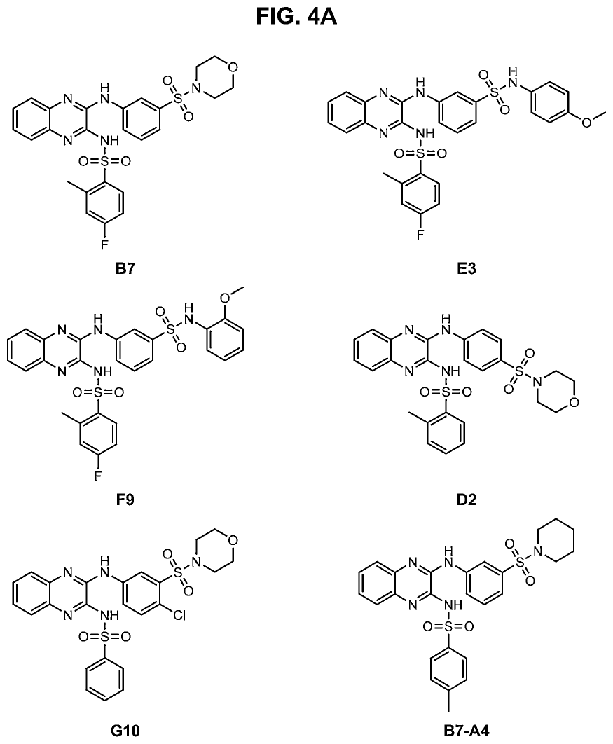

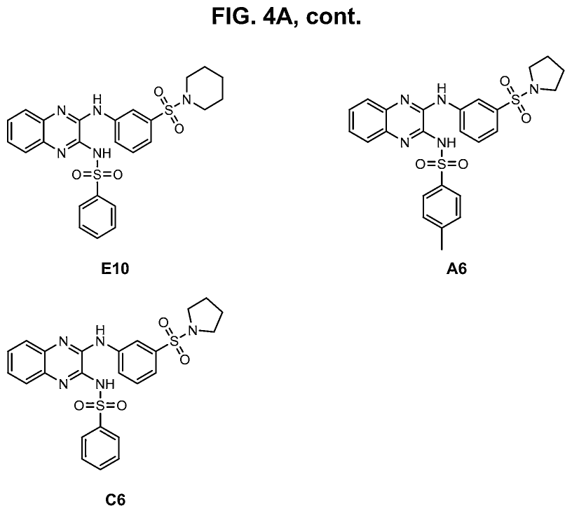

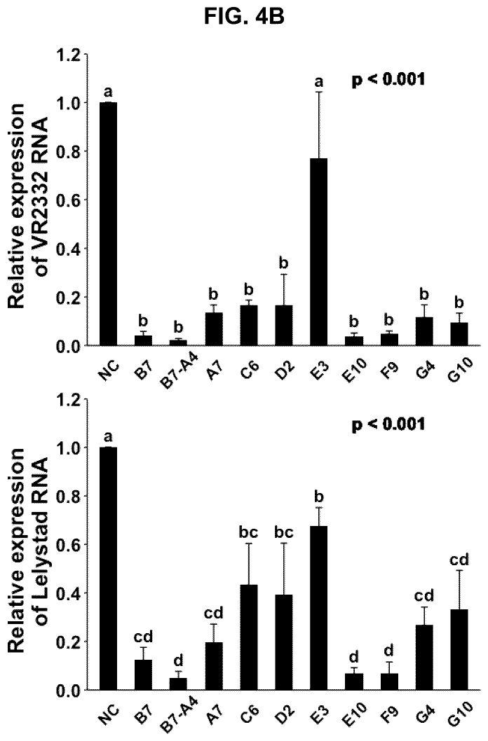

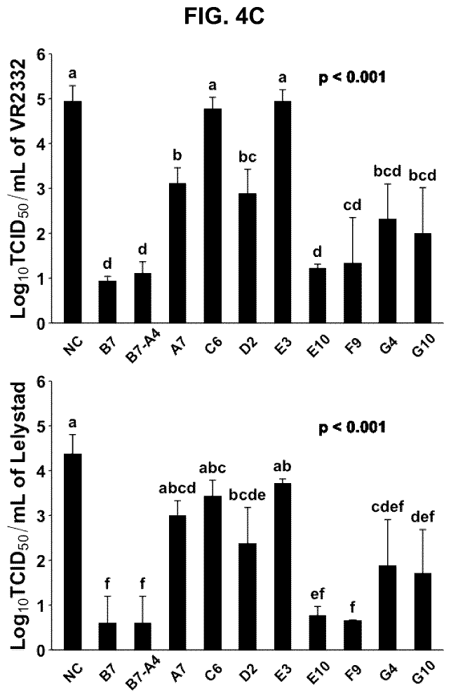

It is important to understand the structure-activity relationship for better design of drugs with improved potency. We therefore asked how the structural changes in different moieties of these compounds may affect their property of anti-PRRSV infection. We organized these chemicals into 2 different groups based on the chemical structure similarities and compared their potency on inhibiting the infection by different strains of PRRSV. In the first group (FIG. 4A), a near complete loss of antiviral-potency was observed on compound E3 (FIG. 4B-C) which replaced the 3-morpholin in B7 by the 4-methoxyphenyl group. Interestingly, however, the antiviral potency did not change at all for compound F9 (FIG. 4B-C), which replaced the 3-morpholin by the 2-methoxyphenyl group (FIG. 4A). Other changes, including the reposition of 3-morpholinosulfonyl in B7 to 4-morpholinosulfonyl group in compound D2 (FIG. 4A), and the removal of 4-fluoro and 2-methyl groups from the benzenesulfonamide moiety in G10 (FIG. 4A), also mildly but significantly decreased the PRRSV infection inhibitory effect for these compounds (FIG. 4B-C). Therefore, in B7 molecule, a moiety topology similar to the 3-(morpholinosulfonyl)anilino is desirable, while side group modifications to the benzenesulfonamtide moiety also exert some effects on the antiviral potency.

The analogue B7-A4 has the replacement of 3-morpholinosulfonyl group in B7 by 3-piperidinylsulfonyl group and shared similar anti-PRRSV activity as B7 (FIG. 4A-C). We also used it as a control to evaluate the impact to antiviral effect by modification of the benzenesulfonamide moiety. Compared with B7-A4, compound E10 with the reposition of 4-methyl group to 3-methyl on the benzenesulfonamide moiety (FIG. 4A) showed no change in antiviral potency (FIG. 4B-C). However, complete removal of the methyl group in compound G4 (FIG. 4A) significantly decreased its antiviral potency (FIG. 4B-C). Therefore, these data confirmed that 2- or 3-methyl modification of the benzenesulfonamide moiety is also important for the antiviral effect of B7. Furthermore, similar to observed above for the importance of 3-(morpholinosulfonyl)anilino moiety, the replacement of piperidin by pyrrolidine group in compound A7 (FIG. 4A) caused a significant drop of its antiviral potency (FIG. 4B-C).

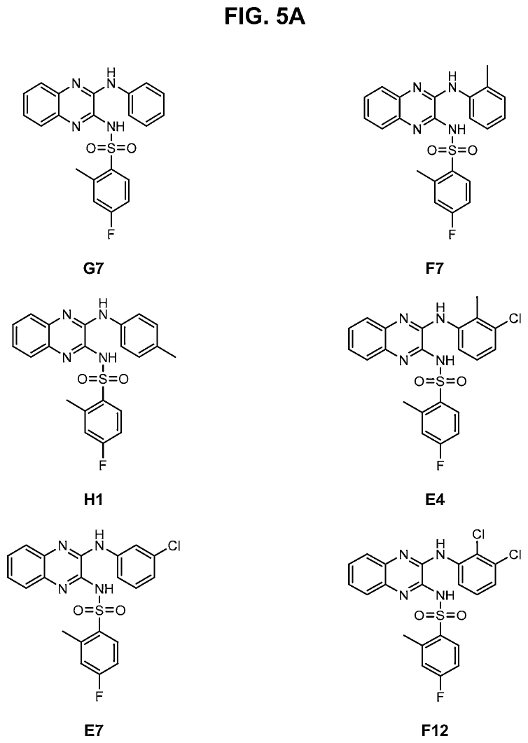

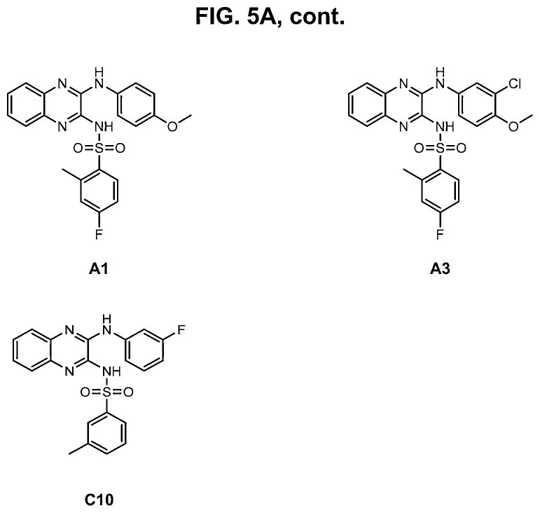

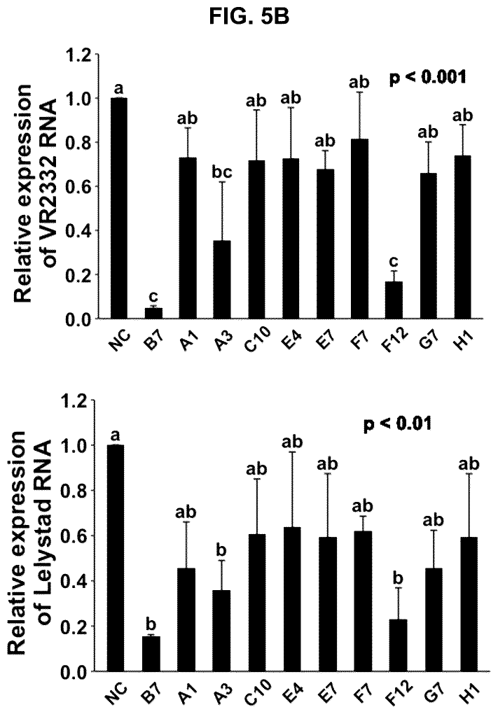

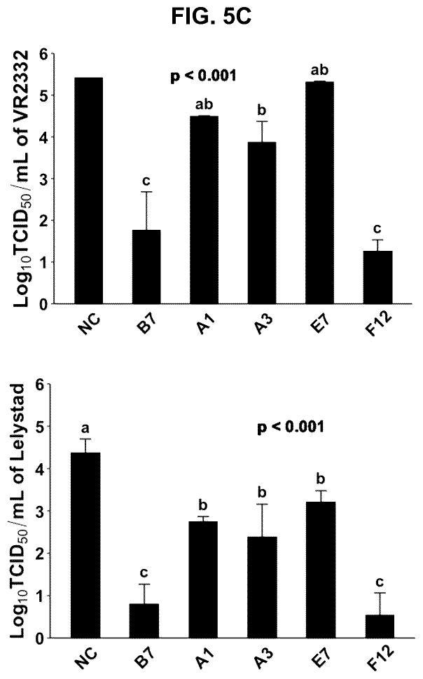

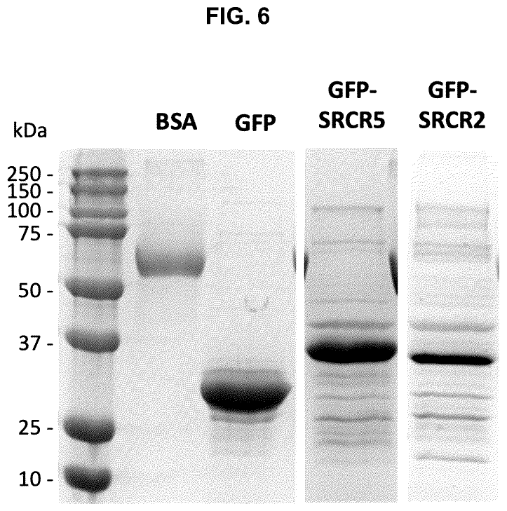

We further asked whether in these compounds, the 3-morpholinosulfonyl group could be replaced by other modifications without a significant decline in the anti-PRRSV potency. Compound G7, which does not have a 3-morpholinosulfonyl group, showed a much lower antiviral effect compared with B7 (FIG. 5B-C). Compounds F7, H1, E4, E7, F12, C10, A1 and A3 have a methyl, chloro, fluoro or methoxymethyl group in place of the 3-morpholinosulfonyl group (FIG. 5A). Among these compounds, only F12 with two chloro group substitutions, and A3 with one chloro and one methoxymethane group substitutions exhibited a significantly increased antiviral effect, while all non-polar modifications, single chloro or single methoxymethyl substitution showed no effect on improving the antiviral potency compared with G7 (FIG. 5B-C). Noticeably, compound F12 exerted an antiviral potency nearly identical to B7 (FIG. 5B-C). Therefore, these data demonstrate that the 3-morpholinosulfonyl group of B7 compound can be replaced by other chemical modifications, including multiple chloro substituents without affecting its inhibitory potency.

PRRS is one of the most economically significant global porcine diseases affecting the swine industry globally. While there are vaccines to control the disease, these are not widely protective due to the constant evolution of the virus. There are no effective antivirals that can be utilized to aid in the control of PRRS. The interactions between PRRSV minor envelope glycoproteins with CD163 result in successful infection. The CD163-SRCR5 domain is involved in receptor recognition by PRRSV. However, it is unclear how each of the glycoprotein-CD163 interactions contributes to efficient PRRSV infection. Combining the BiFC and viral infection assays, we found here that compounds that inhibit the PPI between PRRSV-GP4 and CD163-SRCR5 can efficiently block the PRRSV infection. Also, compared with the GP4/SRCR5 BIFC, which is highly selective on compound with potent activity, the GP2a/SRCR5 BiFC analysis identified a wider range of compounds with various efficiencies against PRRSV infection. This includes compound F12 that does not effectively interfere with the PPI between GP4/SRCR5 (FIG. 1D). These data thus confirm that GP4 and GP2a are both involved in their respective interaction with CD163 in order to establish productive viral infection to host cells.

Although we previously reported the interference of the PPI between PRRSV glycoproteins and porcine CD163-SRCR5 domain by B7 and B7-A4 small molecules, no direct evidence was provided for the specific ligand-protein interaction by these compounds. In this study, we demonstrated that the CD163-SRCR5 domain can interact directly with B7, B7-A-4, and the other newly identified compounds that potently inhibit PRRSV infection. Molecular docking further indicates that the B7 molecule interacts directly with P447 and S554 in SRCR5, which are unique for SRCR5 among the nine SRCR domains of CD163. This correlates well with our MST results that B7 did not interact with SRCR2, and highlighted the SRCR5-targeting specificity by the B7 analogue small chemicals identified here.

Our SAR study provided evidence that the inhibitory function of B7 and its analogues is correlated with the topology of 3-(morpholinosulfonyl)anilino moiety, and additional side group modifications to the benzenesulfonamide moiety in B7. More interestingly, we found that although complete removal of the 3-morpholinosulfonyl group in compound G7 resulted in obvious loss of the antiviral activity compared with B7, this can be rescued by two chloro substituents in compound F12. The addition of chloro groups may rescue the antiviral potency of the compound by changing the topology of anilino moiety, or simply by improving its polarity. Overall, our findings by SAR analysis provided useful functional information and clues for downstream modification of these compounds to further improve the antiviral potency and bioavailability.

Currently, there is no broadly effective vaccine developed to prevent PRRS and control the huge economic loss in global pork industry. This study, together with a better understanding of the PPIs in PRRSV glycoprotein-CD163 recognition, will facilitate the identification of effective antivirals against PRRS. The B7 compound and its new derivatives identified here have the potential to serve as veterinary antiviral drugs if they pass in vivo efficacy test against PRRSV infection and other safety measures. These antivirals have promising activities that can be applied as a critical tool to help control the disease.

PRRS is a global panzootic with huge annual loss to the pork industry but by far with no effective treatment scheme. By using a defined small molecule library, we compared the screening efficiency of two BiFC assays concerning the PPI between CD163-SRCR5 with PRRSV GP2a or GP4 proteins. We found that the BiFC screening of the PPI between SRCR5 and GP4 successfully identified a selective group of compounds with high anti-PRRSV potency, while a similar screening of the PPI between SRCR5 and GP2a resulted in a broader range of compounds with various antiviral abilities, including additional compounds with high inhibitory potency. We further confirmed the physical association of these compounds with SRCR5 protein. SAR analysis revealed that the specific topology of 3-(morpholinosulfonyl)aniline moiety and modifications to the benzenesulfonamide moiety improved the antiviral potency in these compounds. Furthermore, we found that that the 3-morpholinosulfonyl group can be replaced by two chloro substituents without obvious loss of antiviral potency. Our study thus provided powerful screening tools and structure-functional information for the development and optimization of veterinary antivirals to prevent/treat PRRSV, especially for the development and optimization of antivirals to prevent/treat PRRSV infection in pigs.

What is claimed:

1. A method for treating porcine reproductive and respiratory syndrome (PPRS) by administering to a subject in need thereof a therapeutically effective amount of a compound, or pharmaceutically acceptable salt thereof, of Formula (I): 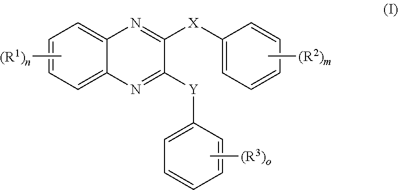
wherein:
X and Y are each independently NR^{a}, O, S, or NR^{a}SO₂;
R¹, at each occurrence, is independently hydrogen, halogen, cyano, nitro, —N(R^{a})₂, —C₁₋₆alkyl, C₁₋₆haloalkyl, —C₂₋₆alkenyl, —C₂₋₆alkynyl, —C₁₋₆alkoxy, or —C₀₋₆alkylCOOR^{a};
R², at each occurrence, is independently hydrogen, halogen, cyano, nitro, —SO₂R^{b}, —N(R^{a})₂, —C₁₋₆alkyl, —C₁₋₆haloalkyl, —C₁₋₆alkoxy, —C₂₋₆alkenyl, —C₂₋₆alkynyl, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R³, at each occurrence, is independently halogen, cyano, nitro, —N(R^{a})₂, —C₁₋₆alkyl, —C₁₋₆haloalkyl, —C₂₋₆alkenyl, —C₂₋₆alkynyl, —C₁₋₆alkoxy, —C₀₋₆alkyl-(COOR^{a}), —C₀₋₆alkyl-(C₃-C₇cycloalkyl), —C₀₋₆alkyl-(heterocycloalkyl), —C₀₋₆alkyl-(aryl), or —C₀₋₆alkyl-(heteroaryl);
R^{a}, at each occurrence, is independently hydrogen, —C₁₋₆alkyl, —C₂₋₆alkenyl, —C₂₋₆alkynyl, —C₀₋₆alkyl-C₁₋₆alkoxy, —C₀₋₆alkylCOR^{a}, —C₀₋₆alkyl-COOR^{c}, —C₀₋₆alkyl-C₂₋₆alkenyl, or C₀₋₆alkyl-C₂₋₆alkynyl,
R^{b}, at each occurrence, is independently —C₁₋₆alkyl, —C₀₋₆alkyl(acyl), —NR^{c}, —NR^{c}-(aryl), —C₀₋₆alkyl-(aryl), —NR^{a}-(heteroaryl), —C₀₋₆alkyl-(aryl), or —C₀₋₆alkyl-(heteroaryl), or —C₁₋₆alkyl-C₁₋₆alkoxy; wherein R_{b} is optionally substituted with substituents such as halogen, cyano, nitro, hydroxy, alkoxy, haloalkyl, oxo (C═O), —C₁₋₆alkyl, cycloalkyl such as —C₃₋₇cycloalkyl, a heterocycloalkyl, a heteroaryl, or an aryl;
R^{c}, at each occurrence, is independently hydrogen or —C₁₋₆alkyl,
n, m, and o are each independently an integer of 0, 1, 2, 3, 4, or 5.

2. The method of claim 1, wherein the compound, or a pharmaceutically acceptable salt thereof, has a structure of Formula (Ia):

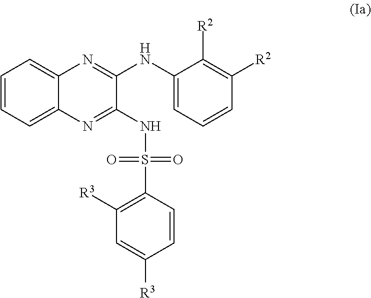

3. The method of claim 2, wherein R², at each occurrence, is chloro.

4. The method of claim 2, wherein R³, at each occurrence, is fluoro or C₁alkyl.

5. The method of claim 1, wherein the compound, or a pharmaceutically acceptable salt thereof, has a structure of Formula (Ib):

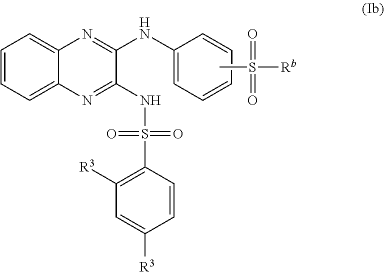

6. The compound of claim 5, wherein R^{b} is

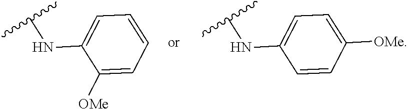 [SMILES: *Nc1ccccc1OC ; *Nc1ccc(OC)cc1]

7. The compound of claim 5, wherein R³, at each occurrence, is fluoro or C₁alkyl.

8. The compound of claim 1, wherein the compound, or pharmaceutically acceptable salt thereof, is selected from the group consisting of:

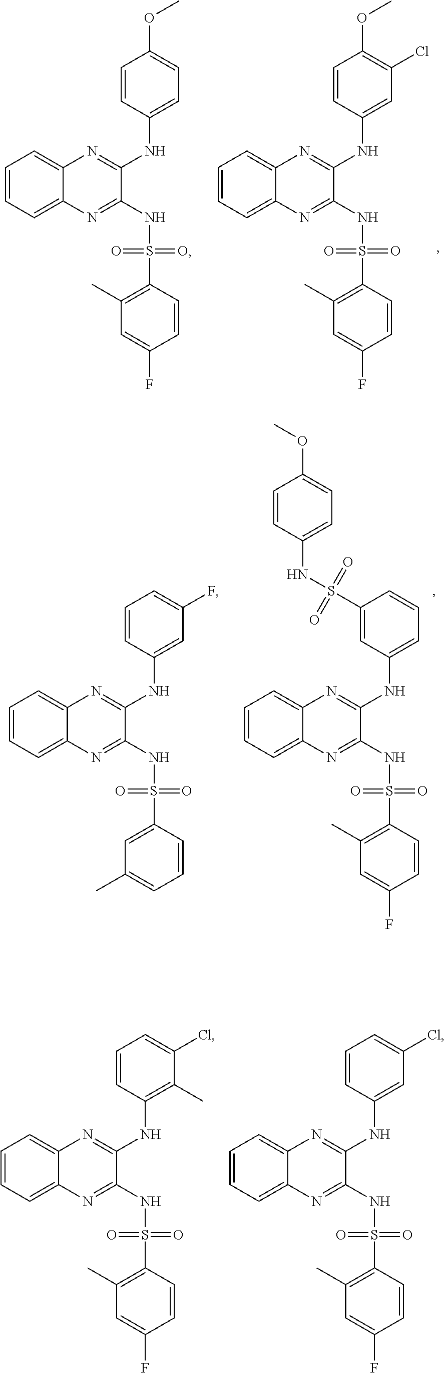

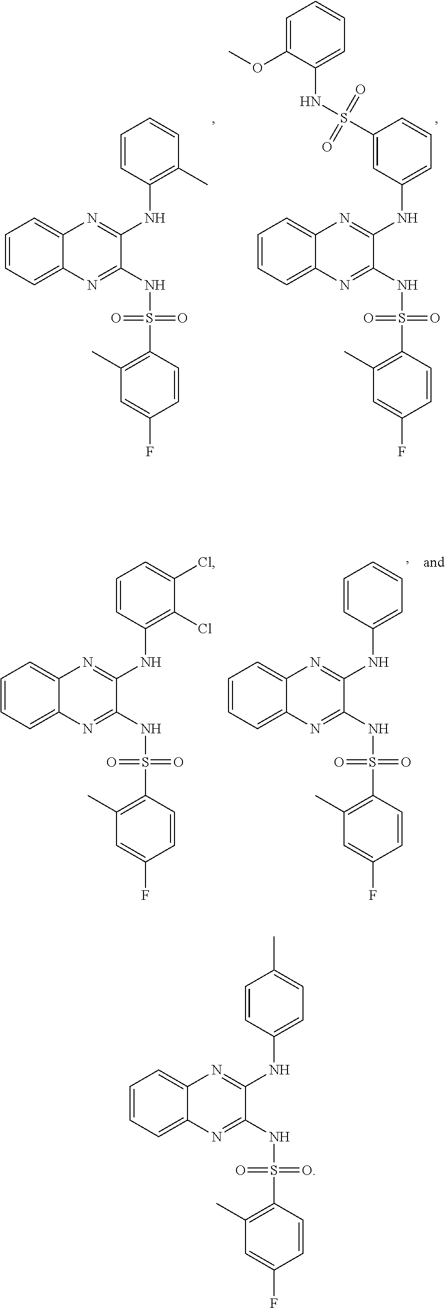

9. The method of claim 1, wherein the porcine reproductive and respiratory syndrome comprises one or more of the following symptoms: fever; lethargy; loss of appetite; vomiting; cough; sneezing; wheezing; labored breathing; high blood pressure; low blood pressure; respiratory distress; depression; cyanosis of the ears, abdomen, or vulva, stillbirths, premature births; abortions; postweaning respiratory diseases; pulmonary edema, or cardiac arrest.

10. The method of claim 1, wherein decreased function of pulmonary alveolar and intravascular macrophages causes the porcine reproductive and respiratory syndrome.

11. The method of claim 1, wherein the compound of Formula (I) is administered to the subject prior to, during, and after infection with an arterivirus.

12. The method of claim 1, wherein the administering is oral, nasal, topical, intravenous, subcutaneous, intramuscular, intravaginal, or intrarectal.

13. The method of claim 1, wherein administering is performed until the disease or disorder is treated, ameliorated, or at least one symptom is reduced.

14. The method of claim 1, wherein one or more chemotherapeutic agents, analgesics, anti-infectives, anti-virals, expectorants, decongestants, anti-fever, or other pharmaceutical agents are co-administered.

15. The method of claim 1, wherein administering the therapeutically effective amount reduces porcine reproductive and respiratory syndrome (PRRS) virus.

16. The method of claim 1, wherein the pharmaceutically effective amount is about 0.5 mg/kg to about 2.5 mg/kg of the compound of Formula (I).

17. The method of claim 1, wherein the subject is a mammal.

18. The method of claim 1, wherein the subject is a pig.

19. A method for treating porcine reproductive and respiratory syndrome (PPRS) by administering to a subject in need thereof a pharmaceutical composition comprising a therapeutically effective amount of a compound, or pharmaceutically acceptable salt thereof, of claim 1; and one or more pharmaceutically acceptable carriers or excipients.

20. The method of claim 19, wherein the one or more pharmaceutically acceptable carriers or excipients comprise buffering agents, solubilizers, solvents, antimicrobial preservatives, antioxidants, suspension agents, a tablet or capsule diluent, or a tablet disintegrant.


SEQUENCE LISTING (8 sequences)

SEQ ID NO: 1 - DNA, 20 bp, synthetic construct
  source 1..20: mol_type = other DNA
aagatgacatccggcaccac

SEQ ID NO: 2 - DNA, 22 bp, synthetic construct
  source 1..22: mol_type = other DNA
ccggcagcataaactcaacctg

SEQ ID NO: 3 - DNA, 20 bp, synthetic construct
  source 1..20: mol_type = other DNA
ggatggccagccagtcaatc

SEQ ID NO: 4 - DNA, 24 bp, synthetic construct
  source 1..24: mol_type = other DNA
tgacgtcatcttcagtcgctagag

SEQ ID NO: 5 - DNA, 20 bp, synthetic construct
  source 1..20: mol_type = other DNA
aaaccagtccagaggcaagg

SEQ ID NO: 6 - DNA, 23 bp, synthetic construct
  source 1..23: mol_type = other DNA
gcaaactaaactccacagtgtaa

SEQ ID NO: 7 - DNA, 20 bp, synthetic construct
  source 1..20: mol_type = other DNA
catcctgggctacactgagg

SEQ ID NO: 8 - DNA, 20 bp, synthetic construct
  source 1..20: mol_type = other DNA
gcttgacgaagtggtcgttg